(19) United States
(12) Patent Application Publication
(10) Pub. No.: US 2022/0414276 A1
(43) Pub. Date: Dec. 29, 2022

(54) DESIGNING TRANSPORTATION AND FACILITIES FOR BIOPROTECTION USING LUMPED ELEMENT MODEL

(71) Applicant: United States of America as Represented by The Secretary of The Army, Alexandria, VA (US)

(72) Inventor: Mark D. Ginsberg, Champaign, IL (US)

(21) Appl. No.: 17/349,544

(22) Filed: Jun. 16, 2021

(51) Int. Cl. G06F 30/12; G16H 40/20; G16H 50/80; G06Q 10/10; G06F 30/20

(52) U.S. Cl. CPC G06F 30/12; G16H 40/20; G16H 50/80; G06Q 10/101; G06F 30/20; G06F 2111/10

(57) ABSTRACT

In one embodiment, a method includes: accepting input data for a design including arrangement of spaces of a structure, operating parameters of a ventilation system, and locations and relative positions of an uninfected individual and one or more infected individuals in the structure with respect to air flowing in the structure and influenced by the ventilation system; calculating an inverse protection factor for the structure using a lumped element model, the inverse protection factor being an inverse of a protection factor which is a ratio of contaminant which the one or more infected individuals exhale in the structure and contaminant which the uninfected individual inhales in the structure; comparing the calculated inverse protection factor to a preset criterion; and if the calculated inverse protection factor fails to meet the preset criterion, changing the design, and repeating the calculating, comparing, and changing until the calculated inverse protection factor meets the preset criterion.

### CROSS-REFERENCE TO RELATED APPLICATIONS

The present application is related to concurrently filed U.S. Patent Application (Attorney Docket Number COE-821A), entitled BIOPROTECTION OF TRANSPORTION AND FACILITIES USING LUMPED ELEMENT MODEL, which is incorporated herein in its entirety by reference.

### STATEMENT OF GOVERNMENT INTEREST

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees.

### BACKGROUND

### Field of the Invention

The present invention relates to apparatus and methods of designing and/or controlling air systems to protect people against disease spread including viral spread.

### Description of the Related Art

This section introduces aspects that may help facilitate a better understanding of the invention. Accordingly, the statements of this section are to be read in this light and are not to be understood as admissions about what is prior art or what is not prior art.

The anthrax attacks of 2001 energized research directed toward reducing health consequences from airborne contaminants by augmenting current heating ventilation and air-conditioning (HVAC) systems. Even during peacetime, interest will continue in improving HVAC components to reduce biocontaminants associated with sick building syndrome.

Currently, national concern is focused on limiting the spread of the SARS-CoV-2 virus. Current recommendations from ASHRAE emphasize using fresh air as weather conditions permit and deprecating the use of recirculated air. A second threat can be the release of airborne biocontaminants from an outdoor source. In that case, the HVAC system should rely on recirculated air as much as possible.

Historically, air movement via HVAC is modeled using an ordinary differential equation (ODE). Up until recently, the resulting ODEs were sufficiently complex that their solutions were approximated via numerical simulation methods. Current HVAC design uses numerical simulation methods of ordinary differential equations to predict approximate performance.

### SUMMARY

Numerical simulations do not yield algebraic expressions describing movement of airborne contaminants within a structure. Indeed, building up a graph describing air quality as a function of any system parameter requires moving the parameter by small increments and slowly building up a graph using one simulation run per data point. For instance, A single CONTAM simulation for a simple structure requires about 6 minutes. Hence a single graph may consume hours of computer time. Even at the conclusion of these simulations, the functional dependence can only be guessed at by curve fitting or other suitable heuristics.

The present invention has been developed to address the desire for a faster method of analyzing HVAC systems in a structure for bioprotection against airborne contaminants and the like. Research and development have led to a novel method of calculating and analyzing a protection factor against contaminants in an HVAC system that enables quick calculations in real time and fast response, in real time based on the calculations, to improve bioprotection of individuals in the structure such as a transportation system/module, building facilities, and the like.

While HVAC is used throughout this disclosure, the relevant feature to bioprotection at this time is ventilation. Heating and air conditioning may be relevant if temperature has an effect on the contaminant. As used herein, accordingly, the term HVAC is not meant to be restrictive; that is, HVAC is configured to affect airflow in a structure including filtration thereof and is not required to be capable of performing all functions of heating, ventilation, and air conditioning. As such, HVAC and ventilation are used interchangeably in this disclosure in that both influence air flowing in a structure, while heating and cooling are features that may be used in situations where they can affect bioprotection.

Heretofore, HVAC engineers have worked out how to address concerns of air quality using many ad hoc and heuristic methods. This is because detailed analysis normally requires the ability to solve differential equations (Navier-Stokes, etc.) at a level of detail beyond currently existing supercomputers. A previous approach involves numerical approximation of a broad class of differential equations that have long been understood to characterize fate and transport of airborne contaminants within a structure and its HVAC system. The current method solves these differential equations outright, and then applies the final value theorem to characterize fate and transport of bio-contaminants. The resulting solutions increase the understanding of how HVAC systems can either enhance, or inhibit, spread of airborne infection.

The algebraic equations in this disclosure predict the dose of contaminant from the infected to the uninfected occupant. Understanding the algebraic structure of these solutions extends the ability to anticipate how airborne infections can be spread in more complex environments such as public transportation.

Embodiments of the present invention provide a method of quickly calculating and analyzing a protection factor against contaminants in an HVAC system in a structure. The quick calculation means fast response in real time to improve bioprotection of individuals in the structure by making changes to the HVAC system and its operation.

The protection factor is a specific figure of merit that describes the effect of the HVAC system and building nearly independent of purely biological quantities, including breathing rates and duration of a contamination event. One can predict the aggregate dose of biocontaminant inhaled by a healthy building occupant.

The protection factor is a ratio of biocontaminant which one or more infected occupants exhale in the structure and biocontaminant which an uninfected occupant inhales in the structure. In the case of external release of contaminant, the protection factor is an asymptotic ratio of outdoor-to-indoor air concentration of particulate matter when the outdoor air is held at a fixed contaminant concentration.

This disclosure presents a way to hand-calculate the protection factor normally derived from differential equations. The protection factor is the quantity of central interest when analyzing fate and transport within the HVAC system. The inverse protection factor calculated via state-space ODEs are solvable quickly by inspection of the HVAC system layout.

Having used the resulting method over a number of HVAC system and building/architectural layouts, what emerged was a “lumped linear” model that made calculation of these results much faster and easier. The lumped-element model (also called lumped-parameter model or lumped-component model) simplifies the description of the behavior of spatially distributed physical systems into a topology consisting of discrete entities that approximate the behavior of the distributed system under certain assumptions. Most real-life situations can be calculated by hand after inspection of the HVAC system and room layout. Many engineers have seen a similar situation when studying electrical schematics. The pedagogically sound way to proceed is to formulate simple rules governing voltage sources, current sources, resistors, inductors, and capacitors. Afterward, one learns rules governing how to hook these items together in series and parallel. With these, two sets of rules, hand calculation of all quantities of interest in a circuit are possible. These lumped results could only be discerned after close inspection of the results obtained by linear algebra and the final value theorem. Hence, the results obtained for this disclosure are a non-trivial extension to the mathematics. i.e., the mathematical derivation presented results that were the exact opposite of the pedagogically sound order.

The present invention advances the science of HVAC system design and control. Key to the success of this apparatus is, among others, the ability to calculate the protection factor against inhalation of contaminants by an uninfected individual in the structure in which the HVAC system operates, in real time, such that bioprotection of individuals in the structure can be provided in real time.

An aspect the present invention is directed to a method for design of a ventilation system for a structure to be occupied by individuals. The method comprises: accepting input data for the design including arrangement of spaces of the structure, operating parameters of the ventilation system, and locations and relative positions of an uninfected individual and one or more infected individuals in the structure with respect to air flowing in the structure and influenced by the ventilation system; calculating an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system; comparing the calculated inverse protection factor to a preset criterion; and, if the calculated inverse protection factor fails to meet the preset criterion, changing at least one of the arrangement of spaces of the structure, or one or more of the operating parameters of the ventilation system, or locations or relative positions of the infected and uninfected individuals in the structure, and repeating the calculating, the comparing, and the changing until the calculated inverse protection factor meets the preset criterion.

In some embodiments, the inverse protection factor is an algebraic expression based on at least one of, some of, or all of (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, (v) transmittance of an air filter used to filter airflow through the space occupied by the uninfected individual, and (vi) breathing rate of the one or more infected individuals.

In specific embodiments, the method further comprises calculating a contaminant dose of the uninfected individual in the structure based on the calculated inverse protection factor, according to Dose=(viri exhaled)/(Protection Factor), where the viri exhaled is an amount exhaled by the one or more infected individuals during a time period in which the uninfected individual is in the structure. The calculated inverse protection factor fails to meet the preset criterion when the calculated contaminant dose is greater than a preset contaminant dose threshold. The preset contaminant dose threshold may be about 3 viri in presence of an infected individual exhaling at approximately 1200 viri per hour.

In some embodiments, the method further comprises, if the calculated inverse protection factor fails to meet the preset criterion, simulating at least one of the following changes to the design: increasing the flow rate R of the ventilation system, decreasing the fraction of air recirculated η, decreasing the transmittance of the air filter, changing the location of the uninfected individual to allow the uninflected individual to breathe air directly from the ventilation system, changing the relative positions of the infected and uninfected individuals so that the uninfected individual is not downwind of the one or more infected individuals, placing a partition to block airflow from the infected individual to the uninfected individual without passing through an air filter, modifying the structure or the ventilation system to block airflow from the infected individual to the uninfected individual without passing through an air filter, or modifying the structure or the ventilation system to produce parallel airflows through the space occupied by the uninfected individual and the space occupied by the one or more infected individuals. The method may further comprise, after the calculating, the comparing, and the changing, physically implementing at least one of the simulated changes to adjust the inverse protection factor to a level that meets the preset criterion for the uninfected individual in the structure having the air flowing therein and influenced by the ventilation system.

Another aspect of this invention is directed to a computer-aided design (“CAD”) system for design of a ventilation system for a structure to be occupied by individuals. The system comprises: a user-interface module configured to display the design and to accept modification to the design, the design being displayed based on input data including arrangement of spaces of the structure, operating parameters of the ventilation system, locations and relative positions of an uninfected individual and one or more infected individuals in the structure with respect to air flowing in the structure and influenced by the ventilation system; a calculation module configured to calculate an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system; and a design module configured to invoke the user-interface module, for making the modification to the design, and to invoke the calculation module upon detecting the modification made to the design. The calculation module is configured to compare the calculated inverse protection factor to a preset criterion. The design module is configured to invoke the user-interface module, if the calculated inverse protection factor fails to meet the preset criterion, to accept a modification to the design to change at least one of the arrangement of spaces of the structure, or one or more of the operating parameters of the ventilation system, or locations or relative positions of the infected and uninfected individuals in the structure, and is configured to make the modification. The design module is configured to invoke the calculation module to calculate a modified inverse protection factor for the structure based on the modification and compare the modified inverse protection factor to the preset criterion. The design module is configured to make one or more additional modifications and performing one or more additional calculations by invoking the user-interface module and the calculation module until the modified inverse protection factor meets the preset criterion.

Another aspect of the invention is directed to a computer program product for design of a ventilation system for a structure to be occupied by individuals, the computer program product embodied on a non-transitory tangible computer readable medium. The computer program product comprises: computer-executable code for accepting input data including arrangement of spaces of the structure, operating parameters of the ventilation system, and locations and relative positions of an uninfected individual and one or more infected individuals in the structure with respect to air flowing in the structure and influenced by the ventilation system; computer-executable code for calculating an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system; computer-executable code for comparing the calculated inverse protection factor to a preset criterion; and computer-executable code for, if the calculated inverse protection factor fails to meet the preset criterion, changing at least one of the arrangement of spaces of the structure, or one or more of the operating parameters of the ventilation system, or locations or relative positions of the infected and uninfected individuals in the structure, and repeating the calculating, the comparing, and the changing until the calculated inverse protection factor meets the preset criterion.

### BRIEF DESCRIPTION OF THE DRAWINGS

Embodiments of the invention will become more fully apparent from the following detailed description, the appended claims, and the accompanying drawings in which like reference numerals identify similar or identical elements.

FIG. 1 shows an example of a schematic of an idealized building with its HVAC system having infected and uninfected individuals in the same room.

FIG. 2 shows an example of a schematic of an idealized building with its HVAC system having infected and uninfected individuals in separate rooms in 2-room parallel HVAC configuration.

FIG. 3 shows an example of a schematic of an idealized building with its HVAC system having infected and uninfected individuals in separate rooms in 3-room parallel HVAC configuration.

FIG. 4 shows an example of series rules for rooms in series HVAC configuration.

FIG. 5 shows an example of parallel rules for rooms in parallel HVAC configuration.

FIG. 6 illustrates the rules for Diagrams 1-3 as examples of different room & HVAC layout and occupant positions in different spaces of a structure.

FIG. 7 illustrates the rules for Diagrams 4-7 as examples of different room & HVAC layout and occupant positions in different spaces of a structure.

FIG. 8 illustrates an example of a diagram and an algebraic equation for placing infected and uninfected individuals in the same space in a parallel HVAC layout.

FIG. 9 shows a public or shared transportation rule.

FIG. 10 shows an example of a seating diagram of a bus illustrating a case of disease spread.

FIG. 11 is an example of a graphical plot illustrating filter efficiency as a function of particle diameter.

FIG. 12 shows Table F1 illustrating filter contribution to the protection factor where the fraction of air recirculated η=0.8.

FIG. 13 shows a flow diagram illustrating one example of a process for calculating a contaminant dose based on the lumped element model.

FIG. 14 shows a flow diagram illustrating an example of a bioprotection control/simulation methodology based on the lumped element model.

FIG. 15 is a block diagram of a bioprotection control/simulation system according to an embodiment of the invention.

FIG. 16 shows a flow diagram illustrating an example of a bioprotection design methodology based on the lumped element model.

FIG. 17 is a block diagram of a bioprotection design system according to an embodiment of the invention.

FIG. 18 depicts an example of a computer system or device configured for use with the HVAC system according to an embodiment of the present invention.

### DETAILED DESCRIPTION

Detailed illustrative embodiments of the present invention are disclosed herein. However, specific structural and functional details disclosed herein are merely representative for purposes of describing example embodiments of the present invention. The present invention may be embodied in many alternate forms and should not be construed as limited to only the embodiments set forth herein. Further, the terminology used herein is for the purpose of describing particular embodiments only and is not intended to be limiting of example embodiments of the invention.

As used herein, the singular forms “a,” “an,” and “the,” are intended to include the plural forms as well, unless the context clearly indicates otherwise. It further will be understood that the terms “comprises,” “comprising,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Embodiments of the present invention provide an Bioprotection control/simulation system which calculates an inverse protection factor for a structure using a lumped element model. The inverse protection factor is calculated based on operating parameters of the HVAC system, arrangement of spaces of the structure, and locations and relative positions of infected and uninfected individuals in the spaces of the structure with respect to air flowing in the structure and influenced by the HVAC system. A contaminant dose of the uninfected individual in the structure is calculated based on the calculated inverse protection factor, arrangement of spaces of the structure, and locations and relative positions of the infected and uninfected individuals in the structure with respect to the air flowing therein and influenced by the HVAC system. If the calculated contaminant dose is greater than a preset contaminant dose threshold, the HVAC system can be controlled, simulated, or designed to reduce the contaminant dose.

### Lumped Element Analysis for Building

A sharp result obtained from the analysis given in this disclosure is that the inverse protection factor calculated via state-space ODEs are solvable by inspection of the HVAC system layout. This revelation, although a welcome surprise, is not without precedent. Scientists and engineers have been exposed to these concepts in the context of reading electrical schematics.

For electrical schematics, one learns two classes of rules. First, the individual components: voltage sources, current sources, resistors, inductors, and capacitors are presented along with short descriptions of how current and voltage are calculated at their connections. Second, the rules for hooking the components in series or parallel are given. First the student is well drilled on how to derive the equations governing a circuit. Only afterwards is it finally stated that for very complex circuits with high component counts, the best practice is to present the circuit to a computer. The computer software, in turn, formulates the resulting analysis as an ordinary differential equation and solves for all unknown quantities.

In the context of conducting the research needed for this disclosure, the equations were developed starting with systems of differential equations. After a sufficient number of cases were solved brute-force, the patterns of what would happen when rooms were hooked together in series and parallel emerged. Here are the results of that analysis.

Many of the results here may seem like “common sense.” However, this mathematical style of analysis is new to this problem space. Here one can derive algebraic expressions for the first time, where previous research in this area relied almost exclusively on numerical simulation, and heuristic methods.

The method of setting up the problems given here are standard and ubiquitous. Some of the inventors' earlier work showed that the exact solutions obtained using transform methods agree with numerical approximations, and therefore inherit the lab validation achieved by numerical approximation. Having the algebraic solutions means that the results obtained with the new methods are far more compact and informative and can be used by personnel with limited training in HVAC engineering.

The present solution method allows one to retain algebraic variables for items such as: the fraction of recirculated air η, the filter transmittance as a function of particle diameter T (d), and the flow rate of the HVAC system R. Their roles are now far less mysterious, and the simple expressions reported below are far more tractable for day-to-day use. This disclosure derives expressions for the most common room layouts within a simulated HVAC zone. By starting with an oversimplified structure and gradually adding complexity over a sequence of examples, several new concepts become clear.

In small structures, including most houses, the HVAC system exhausts air (e.g., combustion products from burning natural gas) through a dedicated exhaust vent. Hence, air is also drawn into the structure through crevices around windows, doors, and the foundation. As buildings increase in size, the volume of air contained grows more rapidly than the surface area of the structure, and hence, the HVAC system is configured to exhaust a fixed fraction of air and draw in a nearly equal volume of “make-up air.” This keeps the building's overall air quality acceptably fresh. This air exchange rate is specified dependent upon building use and typically ranges between 3 changes per hour (e.g., in a small dwelling) and 15 changes per hour (e.g., in a hospital, restaurants, etc.).

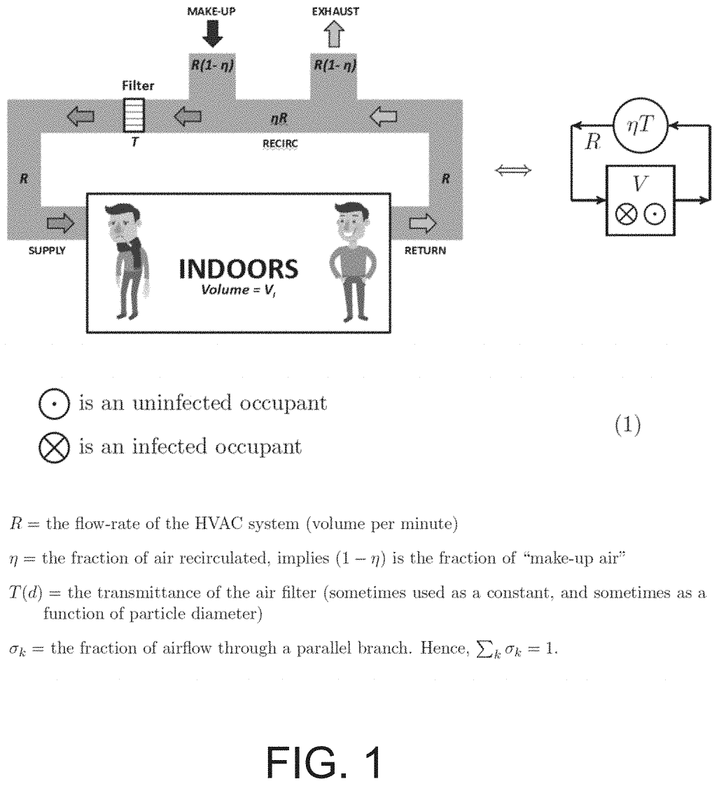

A generic depiction of such a system is given by the diagram in FIG. 1. Here, the figure merely shows an enclosed structure and neglect the architectural layout within. In this diagram the structure is sufficiently large so that the HVAC system is exhausting a fixed fraction of air into the environment and drawing in the same fixed fraction of “make-up-air” to achieve acceptable air quality.

An important concept discovered is that one can capture complex situations with simplified diagrams. Using these diagrams, a practitioner with modest experience should now be able to look at either the diagrams or an actual structure and predict the outcomes as derived in this disclosure. These results are enabling technology leading to many benefits: reducing the complexity of cost/benefit analysis, reducing the risk to building users, and improving public safety.

The mathematical model presented in this disclosure uses the “well-mixed” assumption. This means that each room contains a uniform density of biocontaminants suspended in air. Further, it also assumes that the contaminants cannot settle on building contents including fabrics, and horizontal surfaces. Therefore, the only removal mechanism of bioactive particles is the filter of the HVAC system. For most purposes, these assumptions maximize the expected dose transferred from infected to uninfected building occupants. By erring on the side of maximization, the model is “pessimistic.” Here, responsible engineering analysis should err on the side of pessimism. Hence, the well-mixed assumption is generally helpful in this regard. Further, the well-mixed assumption is also what brings the analysis within range of numerical simulations.

However, the “well-mixed assumption” is less applicable when describing the transfer of virus from an infected occupant to an uninfected occupant when they occupy the same room. Specifically, when an uninfected individual is unfortunate enough to sit directly in the plume exhaled by an infected individual, attention should be paid to the location of all supply registers and return registers to minimize unintended exposure.

On balance, the well-mixed assumption places special emphasis on HVAC design in several different ways, the most important of which is that only the HVAC filter is capable of sequestering biocontamination. The resulting planning numbers for system design are, therefore, valid and conservative.

FIG. 1 shows an example of a schematic of an idealized building with its HVAC system having infected and uninfected individuals in the same room. The schematic of an idealized building has no doors or windows or other leakage. This is used to see the important parts of the analysis in isolation.

For purposes of capturing the correct terms in the model, there are some traditional definitions that are more easily understood if expressed in an altered way. For instance, consider the notion of filter efficiency, defined as the fraction of particles that become trapped in a filter. From the standpoint of ODE modeling, there is more conceptual clarity in the term filter transmittance, defined as the fraction of particulate matter that goes through the filter without becoming trapped. The filter efficiency may be defined as one minus the filter transmittance.

The people in the structure are denoted by the symbols shown in FIG. 1.

⊙ is an uninfected occupant

⊗ is an infected occupant (1)

Quantities of interest include the following:

R=the flow rate of the HVAC system (volume per minute);

η=the fraction of air recirculated, implies (1−η) is the fraction of “make-up air”;

T (d)=the transmittance of the air filter (sometimes used as a constant, and sometimes as a function of particle diameter); and

σₖ=the fraction of airflow through a parallel branch. Hence, ₖσₖ=1.

The ηT in a circle, is a single gain standing for the HVAC system (including the exchange of exhaust and make-up air). This shortcut is what causes the diagrams to capture the mathematics of internal release of biocontaminants. Although the HVAC system shown in FIG. 1 is integrated into a building, it could as well be in another type of architectural structure such as a vehicle (boat, submarine, airplane, spaceship), a space station, or the like.

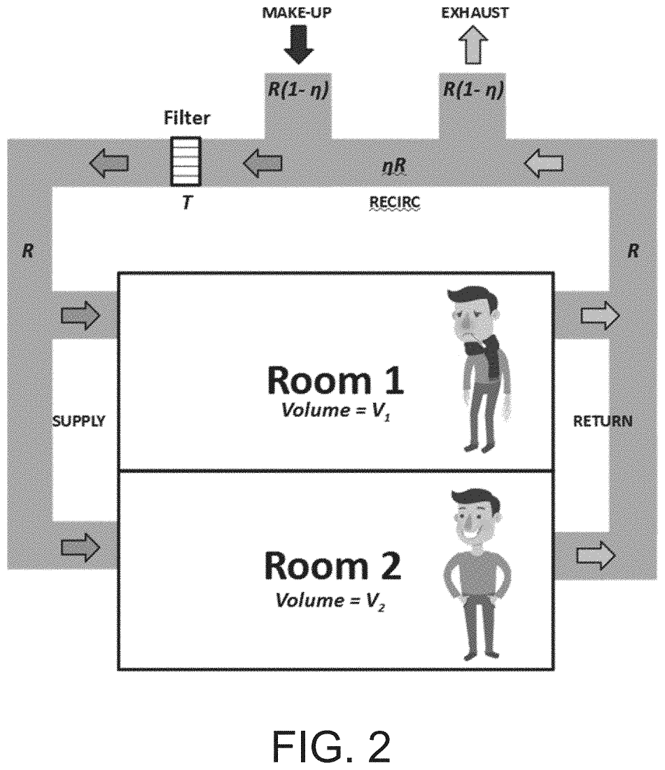

FIG. 2 shows an example of a schematic of an idealized building with its HVAC system having infected and uninfected individuals in separate rooms in 2-room parallel HVAC configuration.

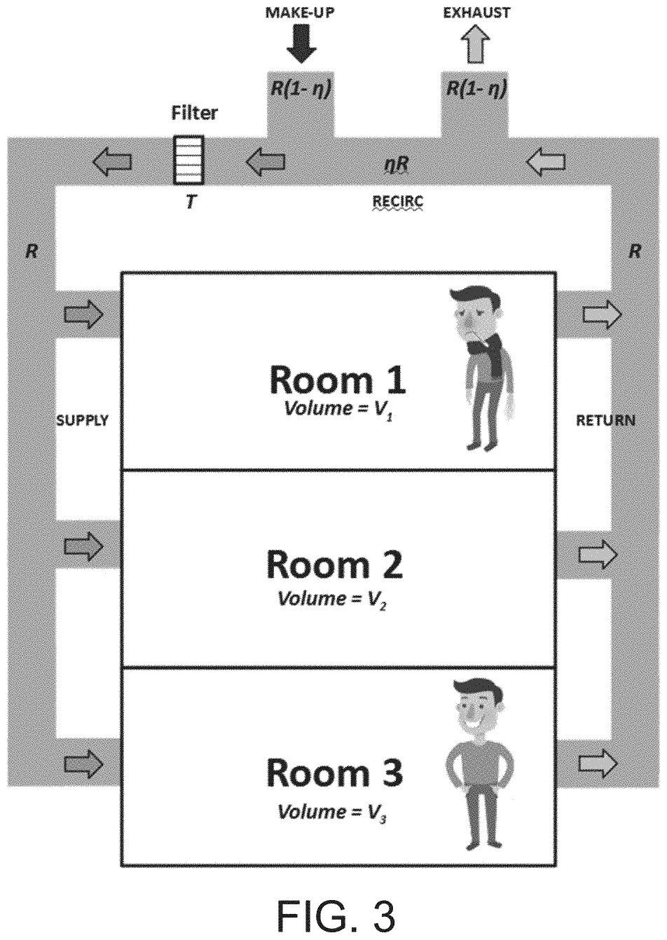

FIG. 3 shows an example of a schematic of an idealized building with its HVAC system having infected and uninfected individuals in separate rooms in 3-room parallel HVAC configuration.

### Rooms (Spaces) in Series

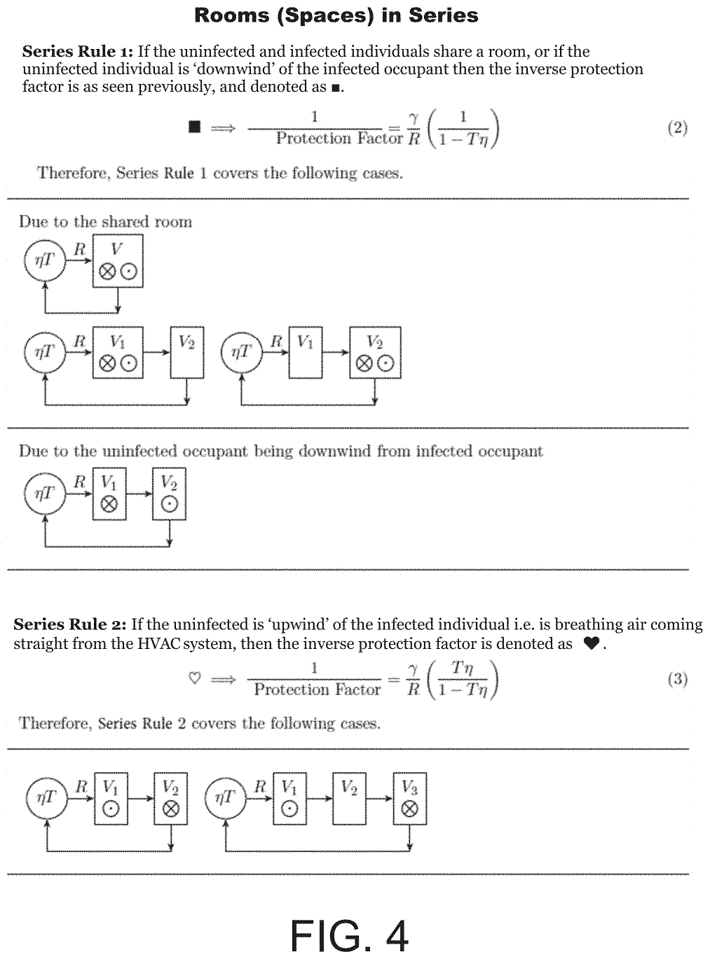

FIG. 4 shows an example of series rules for rooms in series HVAC configuration. Rooms or spaces in series are governed by the following rules.

Series Rule 1: If the uninfected and infected individuals share a room or if the uninfected individual is “downwind” of the infected occupant, then the inverse protection factor is denoted as ▪.

$$◼ ⟹ \frac{1}{Protection Factor} = \frac{γ}{R} ( \frac{1}{1 - T η} ) \qquad ( 2 )$$

The right-hand side of Eq. (2) has two multiplicands. The quantity [symbol] /R is the ratio of the breathing rate of an infected individual to how fast air flows through the HVAC system. As the HVAC system's flow rate increases, so does the protection factor. The second multiplicand, containing terms using T (d) and η, is determined by the filter transmittance and recirculation fraction of the HVAC system.

Series Rule 2: If the uninfected is “upwind” of the infected individual, i.e., is breathing air coming directly from the HVAC system, then the inverse protection factor is denoted as ♥.

$$⟹ \frac{1}{Protection Factor} = \frac{γ}{R} ( \frac{T η}{1 - T η} ) \qquad ( 3 )$$

### Rooms (Spaces) in Parallel

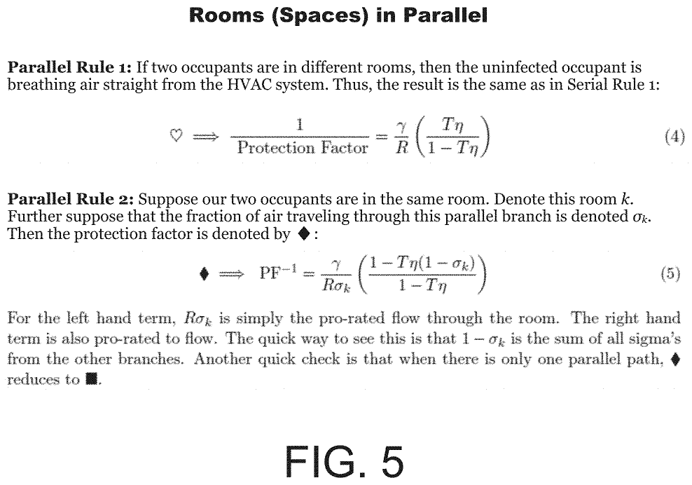

FIG. 5 shows an example of parallel rules for rooms in parallel HVAC configuration. Rooms or spaces in parallel are governed by the following rules.

Parallel Rule 1: If the two occupants are in different rooms, then the uninfected occupant is breathing air directly from the HVAC system. It results in the same rule as Serial Rule 2.

$$⟹ \frac{1}{Protection Factor} = \frac{γ}{R} ( \frac{T η}{1 - T η} ) \qquad ( 4 )$$

Parallel Rule 2: The two occupants are in the same room k. Supposing that the fraction of air traveling through this parallel branch is denoted σₖ, the protection factor is denoted by ♦:

$$◆ ⟹ {PF}^{- 1} = \frac{γ}{R {σ}_{k}} ( \frac{1 - T η ( 1 - {σ}_{k} )}{1 - T η} ) \qquad ( 5 )$$

For the left-hand term, Rσₖ is simply the prorated flow through the room. The right-hand term is also prorated to flow. The quick way to see this is that 1−σₖ is the sum of all sigma's from the other branches. Another quick check is that when there is only one parallel path, ♦ reduces to ▪.

The right-hand side of the equations has two multiplicands. The first multiplicand on the right-hand side of the equation is the ratio of the breathing rate of the infected occupant γ divided by the flow rate of HVAC circulation R. In all practical cases, one expects γ«R. The ratio of the breathing rate of an infected individual to the system's circulation rate is a huge effect. As observed, the ratio of R/γ may be slightly greater than 3500. Therefore, it is beneficial if the fan is left on during all times when the building is occupied. This will turn out to be quite helpful and one will see this same multiplicand repeated later. The second multiplicand of the right-hand side of Eq. (5) is due to having both the infected and uninfected occupant in the same room.

In general, the second term describes the relative positions of an infected versus uninfected individual and the role of room/architectural and HVAC layout. Here one estimates that η is set to a commonly used standard of about 0.8 (almost all buildings run at a fixed value between about 0.75 and about 0.8). Most older HVAC systems use filters that are 1″ thick. A quick perusal of the “big-box” stores and online sources indicates that obtaining these filters of MERV-13 or higher is difficult (i.e., many standard sizes unavailable). Being in the same room or “downwind” of an infected individual knocks this factor down from wherever it is to about 1. There are available data indicating that an infected individual can exhale about 1200 viable viri per hour. In a 10-hour day, that is 12,000. Furthermore, it has been reported that 3 viable viri is enough to sicken an uninfected individual. Therefore, a protection factor of approximately 4000, as derived by these methods, should be considered minimal.

### Room/Architectural & HVAC Layout and Occupant Position

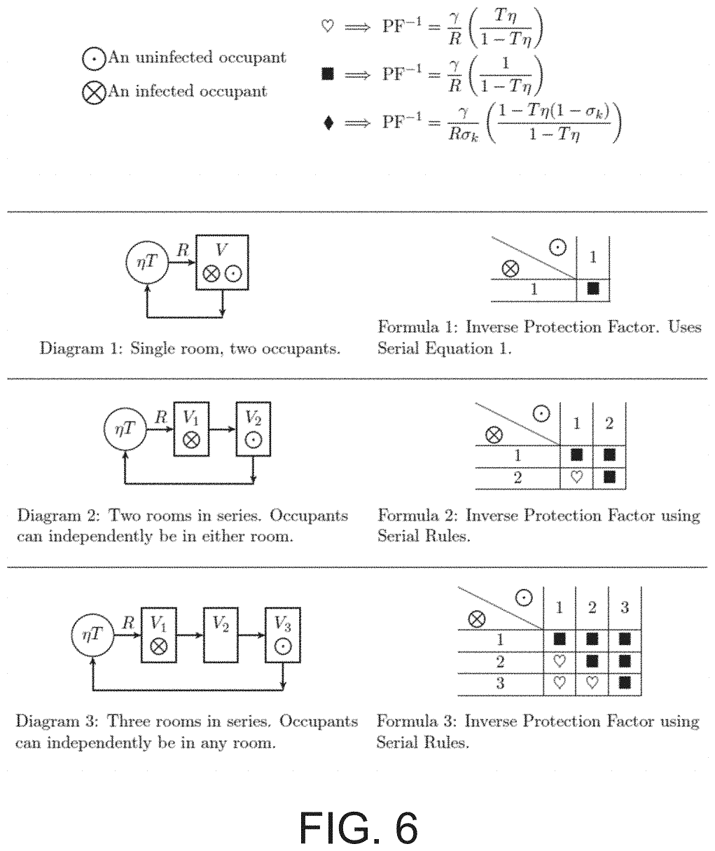

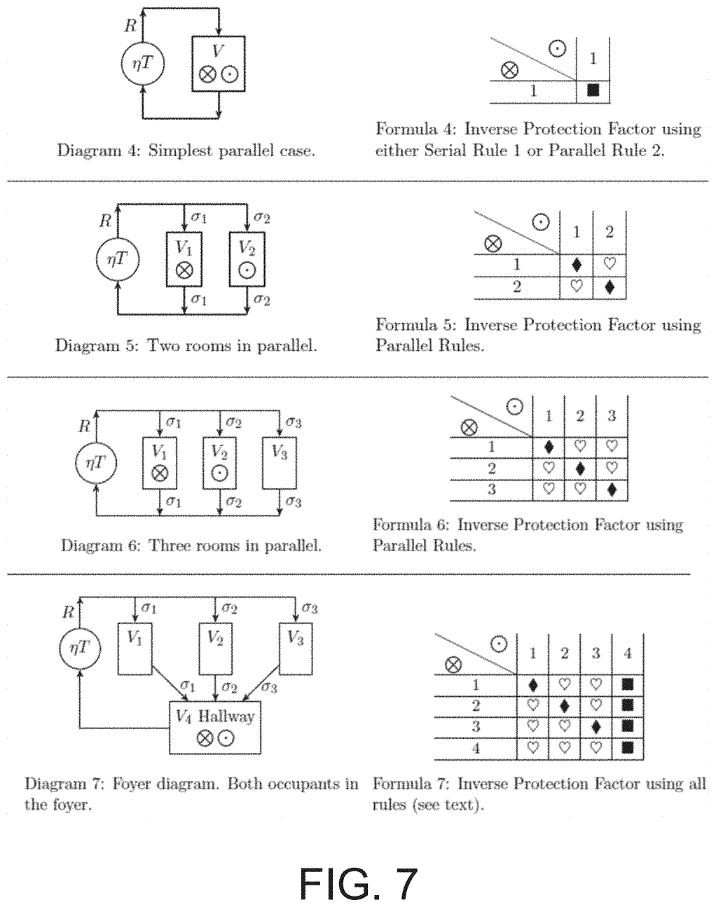

The following summarizes these rules and applies them to various examples of room/architectural & HVAC layout and occupant positions in different spaces of a structure. The examples are illustrated in Diagram/Formula pairs in FIGS. 6 and 7. For each diagram, an uninfected individual and an infected individual are placed somewhere in the structure. The corresponding formula is presented in a table. The table gives all possibilities by listing the placement of the uninfected individual by row (in rooms/spaces 1-4), and the infected individual by column (in rooms/spaces 1-4).

FIG. 6 illustrates the rules for Diagrams 1-3 as examples of different room & HVAC layout and occupant positions in different spaces of a structure.

FIG. 7 illustrates the rules for Diagrams 4-7 as examples of different room & HVAC layout and occupant positions in different spaces of a structure.

There is a surprising result. For example, problems completed for this disclosure, there are three distinct results that occur over a variety of system configurations. These three results are denoted as: ♥, ▪, and ♦.

$$⟹ {PF}^{- 1} = \frac{γ}{R} ( \frac{T η}{1 - T η} ) \\ ◼ ⟹ {PF}^{- 1} = \frac{γ}{R} ( \frac{1}{1 - T η} ) \\ ◆ ⟹ {PF}^{- 1} = \frac{γ}{R {σ}_{k}} ( \frac{1 - T η ( 1 - {σ}_{k} )}{1 - T η} ) .$$

If the uninfected occupant is “upwind” of the infected occupant, then the inverse protection factor is given by a quantity that will be referred to by the expression denoted ♥. In Eq. (3), the presence of the factor Tη in the numerator is what drives this constant to small values.

If the uninfected occupant is either in the same room or “downwind” of the infected occupant, then the resulting expression will be denoted by ▪. In Eq. (2), As one improves the filter, thus driving the transmittance T to near zero, then 1«Tη, hence ▪«♥.

In the three-room scenario of Diagrams 6 or 7, the fraction of air from room n is a convex combination of σₙ, and the constraint that σ₃=1−σ₁−σ₂ has already been substituted.

Consider the example depicted in Diagram 7 and step through the analysis. If one considers both occupants being present in rooms 1 through 3, then the existence of room 4 makes no difference (i.e., one can think of it as a long air duct that does not enter the calculations). Therefore, the upper left 9 entries (square of 3 by 3) in this example should agree with results obtained in Diagram 6. For the right-most column of entries, this signifies that the uninfected individual is “downwind” of the infected individual, or that they are sharing the same room. Therefore, this row agrees with results given in Diagram 2. For the left 3 entries of the fourth row, the uninfected individual is breathing air immediately after processing by the HVAC system, and again by referring to Diagram 2, the result is obtained. These results were also independently obtained using matrix methods.

In practice two things become evident. First, if a particular HVAC zone is filled with byzantine airflow patterns through differing rooms from supply registers to return registers, keeping doors open is the way to keep airflow rapid and may improve the protection factor. Second, keeping different HVAC zones closed from one another is a way that may help to clarify the overall situation.

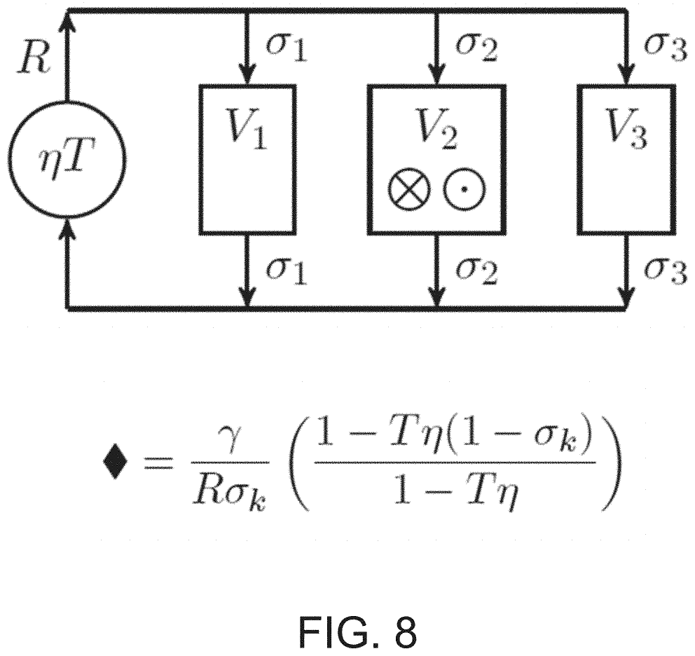

FIG. 8 illustrates an example of a diagram and an algebraic equation for placing infected and uninfected individuals in the same space in a parallel HVAC layout. That is, both occupants are sitting in a room that is in parallel with other rooms which are empty. In this case, the result should look analogous to ▪ but there should be adjustments based on the room only getting a fraction σₖ of the flow. Here, one denotes the result ♦.

$$◆ ⟹ {PF}^{- 1} = \frac{γ}{{Rσ}_{k}} ( \frac{1 - T η ( 1 - {σ}_{k} )}{1 - T η} )$$

where both occupants are in room k. Conceptually, one can think of this as exhaling virus in one room, and even if the filter does not absorb it in the first pass through the filter, the virus can be unavailable for inhalation simply because it could continue to travel in loops through the diagram that do not include the uninfected occupant. Here it is worth noting that the results ▪ and ♦ only differ in two respects. The fraction of flow going through room k is σₖ. Hence the lead term is made somewhat larger by the presence of σₖ. Second, in the limit that the filter transmittance is small, one expects that 1>>Tη(1−σₖ) (recall that 0<σₖ<1 and 0<η<1). So, the reduced flow through the room in question is the dominant effect. (Quick outline of a proof: If there was a crossover point, one would have σₖ=1−Tη(1−σₖ), this implies Tη=1 which never occurs by definition). Therefore, one expects the outcome of ♦ to be worse than ▪.

There are limitations to this analysis. The model is based on immediate dispersal of exhaled contamination throughout the room volume. In real-life, with both occupants in the same room, there can be an undispersed transmission from the infected to the uninfected individual. For purposes of these calculations, the assumption is that “social distancing” of six feet is enough to disperse contaminant and make the approximations valid.

### Lumped Element Analysis for Transportation

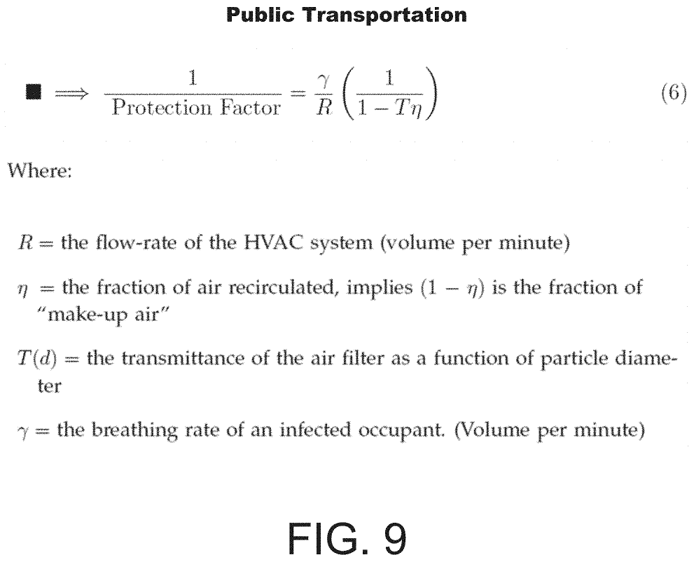

FIG. 9 shows a public or shared transportation rule. It applies to multiple individuals in a transportation system or module.

$$■ ⟹ \frac{1}{Protection Factor} = \frac{γ}{R} ( \frac{1}{1 - T η} ) \qquad ( 6 )$$

The right-hand side of Eq. (6) has two multiplicands. The quantity γ/R is the ratio of the breathing rate of an infected individual to how fast air flows through the HVAC system. In most cases, one expects γ«R. As the HVAC system's flow rate increases, so does the protection factor. The second multiplicand, containing terms using T (d) and η, is determined by the filter transmittance and recirculation fraction of the HVAC system.

Understanding the two multiplicands embedded in the right-hand side of Eq. (6) is immediately useful in understanding problems that arise when discussing viral spread in various modes of public transportation. The first multiplicand on the right-hand side of the equation is the ratio of the breathing rate of the infected occupant γ divided by the flow rate of HVAC circulation R. The second multiplicand of the right-hand side is due to having both the infected and uninfected occupant in the same transportation space.

### Subway Example

As a first example, consider the article by A. Rogers, “How does a virus spread in cities? It's a problem of scale.,” Wired, May 2020. The author raised an unresolved question regarding analysis of the effect of the New York subway system on the spread of COVID-19. Initial conjectures held that even though usage had fallen to 20% of normal, the enclosed spaces in such systems would cause spread. This was directly contradicted by epidemiological data showing that infection density did not increase based on proximity to subway lines. Yet, the simple observations just made about the structure of Eq. (6) are enough to formulate reasonable conjectures regarding this apparent contradiction.

Subways cause immense airflow in several different ways. First, air pushed ahead of a train would cause immense drag, except that the high pressure in front and low pressure left behind are alleviated by street level air vents. Hence there is immense airflow as the trains pump this air out of and then into the tunnels causing a breathing effect. Second, the large exchange of passengers at each station bring with them plenty of air from the station. Third, older cars are very leaky allowing additional airflow through the train car, where newer cars may be well sealed but typically have an aggressive HVAC system to maintain temperature. Fourth, and less appreciated, is that when the trains leave the heart of the city, the balance of the track is elevated above ground. Hence, both on the platforms waiting for trains and within the trains themselves, there is an immense air flow rate that keeps the first term of the equation contributing to a sizable protection factor. Direct transmission from person-to-person in an overpacked train-car is beyond the scope of analysis here.

### Bus Example

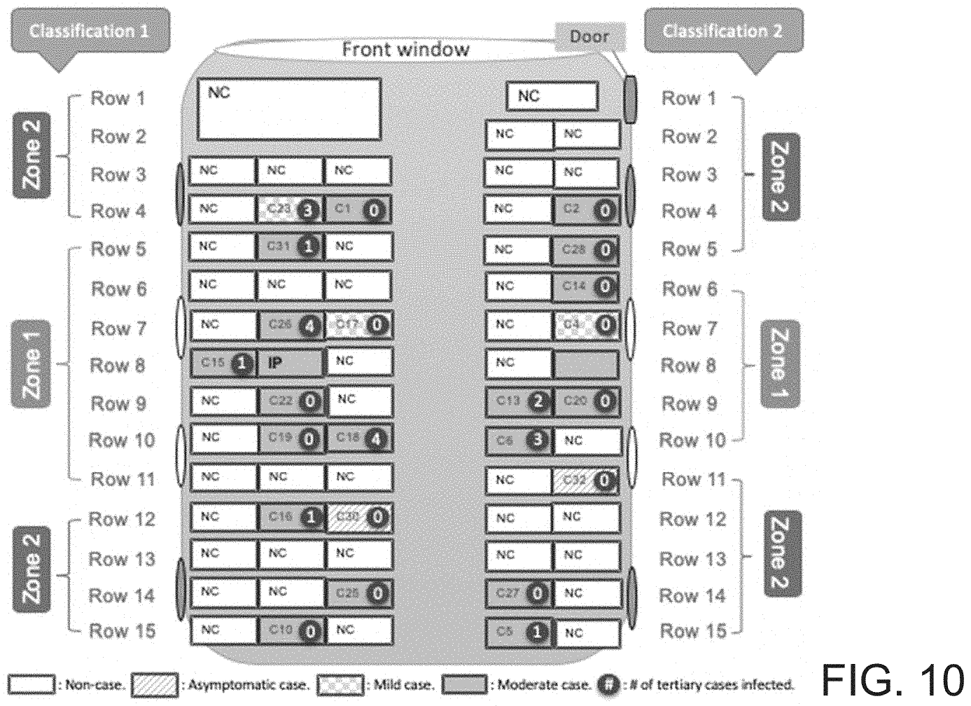

FIG. 10 shows an example of a seating diagram of a bus illustrating a case of disease spread. The initial infected passenger (IP) in seat 8B seemed to spread the virus to many others on her bus, designated by different shadings for asymptomatic case, mild case, and moderate case. The pattern of infected passengers shows distinct signs of the recirculated air blowing from left to right. Buses should be an ideal situation where the HVAC system can be selected to only use fresh air, and the HVAC system, if run constantly, will have a high enough flow rate to limit spread. The diagram shows the seating of other passengers present and identifies those presumably infected during the trip. Hence, where the filtration system of a vehicle has poor filter efficiency, recirculation is not recommended.

### Strategy for Public Transportation

The results show that most transportation systems have protection factors that are dominated by airflow (as opposed to air filtration); the transportation systems' filter quality helps but is less important than in fixed structures. As the HVAC system's flow rate increases, so does the protection factor.

Transportation systems have a natural form of protection available. As vehicles move, they should take advantage of the airflow generated to minimize use of recirculated air and maximize fresh airflow to the riders. However, this does not overcome direct person-to-person transmission. Therefore, social distancing and masks to inhibit direct droplet spread are still advisable. As will be shown in the detailed analysis given, it is best if seating is arranged so that occupants are not effectively downwind of one another.

### HVAC Filters

Filters pull contaminants out of the air using several distinct mechanisms. There are four basic mechanisms: sieving, impaction, interception, and diffusion. The descriptions and dimensions given here vary a lot with the specific filter material and should be taken as no more than useful heuristics.

Sieving: Sufficiently large airborne particles simply cannot pass through the open spaces within the filter material. (This is analogous to putting cooking noodles into a strainer to remove water.) Therefore, particles of a characteristic diameter and larger are easily trapped by the filter material.

Impaction: This describes particles that are sufficiently heavy that they cannot flow along with the air currents that twist and turn to get around fibers in the filter material. As these particles carry too much momentum to follow sharp turns in the airflow, they eventually contact the filter fibers and stick. (This is analogous to a bug being too heavy to flow over a moving car with the slipstream air and striking the car's windshield.) Characteristically this happens to particles that are about 0.9 μm or larger.

Interception: This describes an airborne particle's tendency to simply contact the filter material even when it flows well with the surrounding air stream. Characteristically this happens to particles that are about 0.8 μm or larger.

Diffusion: As particles get smaller than 0.1 μm they collide with individual air molecules resulting in Brownian motion. The resulting random jostling causes the particles to move perpendicular to the path of the air stream. These transverse movements become larger as particle diameter decreases. One way to think about this is that the particles have a much larger effective diameter because they get knocked about resulting in larger transverse movement as they get smaller.

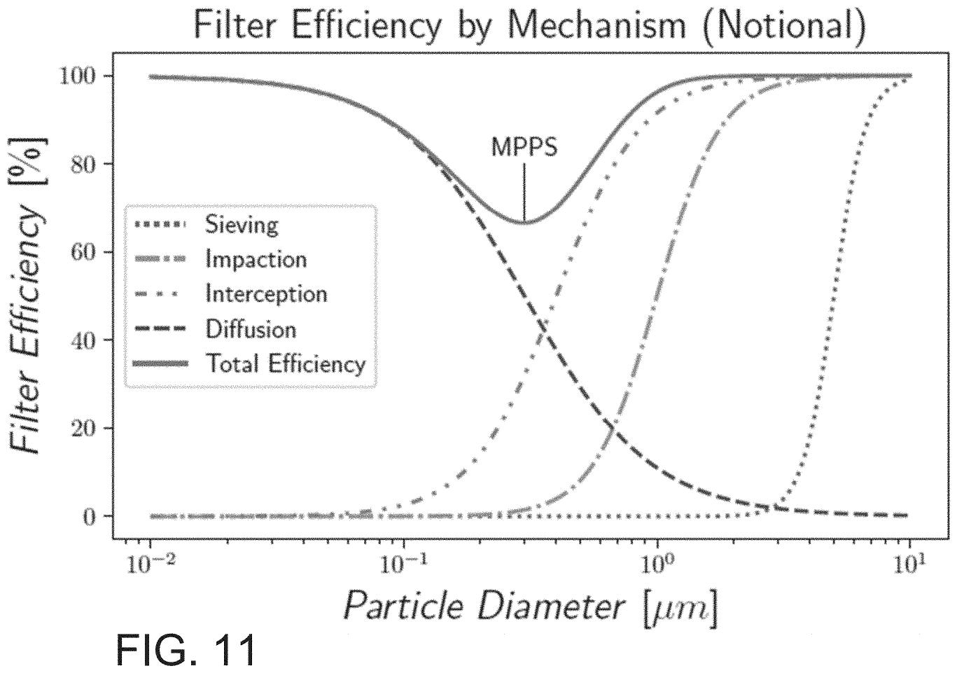

FIG. 11 is an example of a graphical plot illustrating filter efficiency as a function of particle diameter. It depicts the effects of the above-described mechanisms. The solid line represents total efficiency. Maximum penetrating particle size (MPPS) is labeled.

Filter performance is commonly described using the term filter efficiency. This is the probability that a particle entrained in an air steam will be sequestered in one pass through a filter. It is typically given as a percentage, i.e., a filter efficiency of 0% means that the particle of interest will flow directly through the filter and not become trapped. A filter efficiency of 100% means that the particle of interest will definitely become trapped in the filter.

As discussed in the filtration mechanisms above, filter efficiency is dominantly a function of particle diameter. The effect of each of these mechanisms is depicted in FIG. 11. It is noted that the total filter efficiency has a small dip centered around ≈=0.3 μm. This dip and its location are very characteristic across many different filter types. Many filters are specified by the filter efficiency at ≈=0.3 μm. Indeed, filter ratings reflect that there is typically a single dip on the graph of filter efficiency, as an example the MERV (minimum efficiency reporting value) rating directly acknowledges this dip by naming convention. Once filters are 99.97% efficient, they are classified using slightly modified criteria and are rated as HEPA (high-efficiency particulate air).

The filter transmittance is defined as “1—filter efficiency.” This definition yields a very useful mathematical short-cut. Suppose there is one airstream flowing through two air filters in succession. The filters are named a and b. Suppose further that one knows the filter efficiency for each of these, correspondingly named Eₐ and E_{b}. The aggregate filter efficiency of these filters in series is rather cumbersome. Here, E_{T} is the total efficiency in series. Then:

E_{T}=1−(1−Eₐ)(1−E_{b}).

Whereas if one had started with the concept of filter transmittance and named the corresponding quantities Tₐ, T_{b}, and T_{T} respectively, then the relationship would be much simpler:

T_{T}=TₐT_{b}

### Spread of Infection

Given the lack of information on the infectivity of SARS-CoV-2, currently this disclosure relies on other results from peer-reviewed literature that report on other viruses. For an infected individual within the building envelope, it would be handy to know how many viable viri they exhale in a minute. The upper bound of what has been documented is 1200 per hour. For a healthy individual inside the building envelope, typical tidal breathing at rest (called respiratory minute volume) is in the range of 5 L min⁻¹ to 8 L min⁻¹ and will suffice for the moment. For particle diameters in the range of 0.15 μm to 0.4 μm anyone who inhales them simply does not exhale them again, and in this sense is a pure integrator of such particles in the air they are breathing.

### Example of Calculations Based on the Lumped Element Model

Step 1: Understanding the strategy—Assume social distancing at a 6 ft radius is observed. The number of viri transferred from an infected building occupant to an uninfected one can be estimated at:

$$Dose ( i . e . , Viri inhaled ) = \frac{Viri exhaled}{Protection Factor} \qquad ( F1 )$$

where the viri exhaled is an amount exhaled by the one or more infected individuals during a time period in which the uninfected individual is in the structure.

The strategy here is to hold the “Dose” below a total value of 3 viri in the presence of an infected individual exhaling approximately 1200 viri per hour.

Step 2: Understanding the protection factor—The quantity “1/Protection Factor” or PF⁻¹ (inverse protection factor) takes on one of two values depending on the placement of the infected and uninfected individuals. These two values are denoted as either ▪ or ♥.

$$\begin{matrix}= \frac{γ}{R} ( \frac{T η}{1 - T η} ) \\ ■ = \frac{γ}{R} ( \frac{1}{1 - T η} )\end{matrix} \qquad ( F2 )$$

where:
R=the flow rate of the HVAC system,
η=the fraction of air recirculated (normally 0.8),
T=Transmittance of the air filter (1—filter efficiency), and
γ=an average breathing rate for a human (approximately 8 L min⁻¹).

Step 3: Choose between ♥ and ▪—Fix the placement of the uninfected and infected building occupant. If this is known, choose the value; otherwise use the following rules. For the simple air patterns designed into most structures the following rules suffice:

Rule 0: HVAC zones should be shut off from each other, so that differing pressures do not cause airflow from one zone to the next.

Rule 1: Within a structure, if an uninfected person sits where they breathe air coming from the HVAC system directly, they are protected from an infected individual elsewhere in the structure with a protection factor associated with ♥. However, if they occupy either the same room or a room “downwind” of an infected person, the protection factor is downgraded to the quantity associated with ▪. To be conservative or on the safe side, the expression ▪ also applies if the uninfected individual does not breathe air directly from the ventilation system when an infected individual is in the same structure.

Rule 2: If an uninfected occupant is sitting where the quantity ▪ is in effect (see Rule 1) (e.g., ▪ is in effect if the uninfected individual is downwind of the infected individual or does not breathe air directly from the ventilation system when the infected individual is in the same structure), and the layout of the structure (several paths served in parallel) means that a fixed fraction of the air flows through the room containing the uninfected occupant. If this happens in the k-th room, define the fraction of airflow as σₖ. Then the expression ▪ is still used, with the quantity R is substituted with the expression Rσₖ.

Step 4: Calculating numeric values for ♥ and ▪—Taking a look at the expressions ♥ and ▪ in more detail, each has a left multiplicand of γ/R and a right multiplicand that depends on T and η.

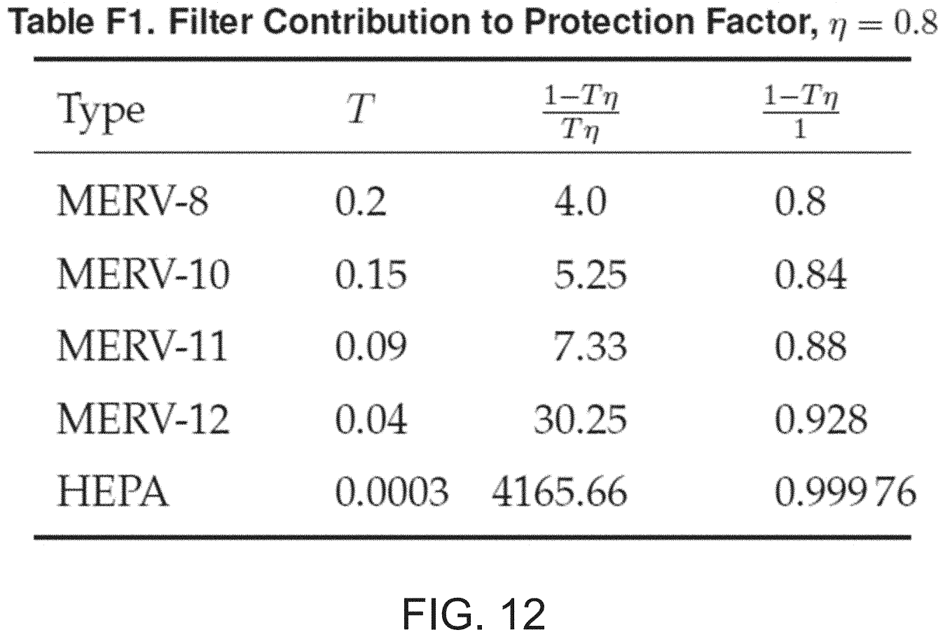

For the right multiplicand, either the HVAC unit will be set up with a specified value for η or one can assume a standard value of η=0.8. If the filter is rated at a specific MERV number, the transmittance can be estimated using the second column of Table F1 or set to T=0.9 if the filter is unrated. FIG. 12 shows Table F1 illustrating filter contribution to the protection factor where the fraction of air recirculated η=0.8.

For the left multiplicand, in the United States, almost all HVAC fans are rated in units of cubic feet per minute (abbreviated cfm). If so, use the following:

$$\frac{γ}{R} = \frac{1}{3.54 \times ( R in cfm )} \qquad ( F3 )$$

Step 5: Calculate the expected dose—What happens in, say, a ten-hour workday? Using Eq. (F1), the uninfected individual receives a dose of approximately

$$Dose = 10 ℏ \times 1200 \frac{viri}{ℏ} \times ( or ■ ) \qquad ( F4 )$$

With a bit of practice what emerges is that many of this full set of calculations can be carried out using Eqs. (F1) to (F3) and Table F1. These all fit easily on an index card.

As can be seen from Eq. (F4), the longer the uninfected individual is in the building, the more risk there is of inhaling an infectious dose. As such, a valid alternative is to minimize the amount of time an occupant is present.

### Cost Benefit Analysis for Buildings

For a modest sized structure (about the size of a house), the following analysis applies. Given that the average cost of a day in a hospital is about $2000, and much more for specialized care required for COVID-19, prices for HVAC upgrades are quite small. The following observations can be made. For a 1″ thick air filter, MERV-8 is about $2.50 with MERV-12 $18.00 (or ≈=111 filter upgrades per hospital day). For a 2″ thick air filter, MERV-8 is about $13.34 with MERV-13 $29.34 (or ≈=66 filters per hospital day). Leaving HVAC air fans running for an extra 6 hours per day is also cheap. At $0.1285 per kW h and with a 1000 cfm, ⅓ hp fan consuming 600 Watts, this is 3.6 kW h d⁻¹. One obtains $0.47 per 1000 cfm per day.

As structures increase in size, the points listed above can be prorated to achieve reasonable estimates of cost versus benefit. It is worth noting that flow rates for larger structures are normally calculated using a combination of floor area, plus a fixed allowance per building occupant. The equations here suggest that the resulting protection factor should be considered as well.

For critical infrastructure (e.g., the critical care unit CCU of a hospital), if upgrades to the filter shelf and surrounding duct work are too expensive, there are still alternatives. Temporarily replacing the applicable section of HVAC system with an external temporary unit that implements improved air purification may be the best alternative.

### Observations

This disclosure presents, for the first time, a way to hand-calculate protection factor normally derived from differential equations. These results are used to show that most transportation systems have protection factors that are dominated by airflow, and that their filter quality helps, but is less important than in fixed structures. As the expected efficacy of these HVAC systems are summarized by equations, the trade-offs implicit in their design are much more easily explained. The disclosure also presents sufficient analysis that those interested in altering existing vehicles and rolling stock with supplementary panels can directly demonstrate the cost effectiveness of their designs.

Protecting personnel from biocontamination is a life-safety issue. The analysis given in this disclosure solves a specific class of differential equations describing fate and transport of biocontamination that are normally implemented via numerical approximation. Computer implementation of the new solutions are many thousands of times faster than their standard numerical approximations. Hence, the newer solutions can serve as an enabling technology for many new applications. These include building and HVAC design, real-time bioprotection, bioprotection for mass/shared transportation, and smart bases for military or the like.

In terms of building and HVAC design, current building and HVAC designs can now be modified with safety enhancements that inhibit biocontamination both from internal and external release. Their performance can be predicted more accurately (and far more cheaply) during the design phase. In terms of real-time bioprotection, there has been heavy investment in the older numerical approximation methods requiring thousands of times more computational effort. Hence only the very largest structures take advantage of near-real-time analysis of sensor-driven measures of air quality. These control systems can now be implemented in a much more cost-effective manner and, hence, can also be extended to smaller structures.

For transportation, HVAC system designs for transportation can be modified to protect occupants from biocontamination, thereby enhancing safety. For smart military bases or the like, current concepts emphasize adapting to changing threats. HVAC systems regulate temperature, humidity, air quality, and air safety by means of feedback control. In principle, there are no technical obstacles to coordination of these control systems on an installation-wide basis. Hence the installation can justify such expenses both to save energy and enhance safety.

### Recommendations

There arises a natural conflict between the protection strategy required for internal release (e.g., an infected occupant exhaling SARS-CoV-2 virus) and external release (e.g., an attack directed toward feeding contaminant to the intake vents of an HVAC system). For internal release, it is best policy to recirculate as little air as possible from within the structure. By contrast, protecting from external release requires maximizing use of recirculated air, thus minimizing use of external contaminated air. For the immediate future, facilities worldwide currently exist in an environment of slow attack via internal release. As such, minimizing use of recirculated air is an excellent option for the moment. However, the fraction of recirculated air should be thought of as flexible (as opposed to permanent), as the threat environment will inevitably change with time.

The following recommendations are now understood quantitatively, rather than by qualitative heuristics available previously. Some of these recommendations are simply not given in any other source.

### Items that can be Addressed almost Immediately at Low Cost

HVAC units with filters should be set up to run the fan continuously whenever personnel are present. And for an additional time afterwards depending upon the air-change rate.

Upgrade air filters to the best rating commonly available in their form factor (i.e., that fit the current filter shelf). These may need more frequent replacement at first.

Seal edges of filters to reduce bypass.

Be rigorous in changing air filters at intervals specified by the manufacturer.

Other sources (e.g., ASHRAE) recommend having no-recirculation of building air when weather permits. For certain situations, this should be weighed against the increased vulnerability to external sources of contamination (e.g., a deliberate attack).

For applicable system geometries, modify positioning (seating or workstations) of personnel present most of the day.

Where possible, assure that each occupant is breathing air from the HVAC system directly and not exhaled by another occupant.

Bias placement toward supply registers.

Bias placement away from return air registers.

Seating in the corner of a room is not recommended.

Additional partitions can block direct flow of biocontamination from one occupant to another (e.g., use partitions to move from the case of two occupants of the same room to the parallel case, i.e., this blocks air flow from one occupant to another without passing through an air filter). When possible, these should be installed parallel to airflow to avoid generating attached vortices.

Doors and partitions that separate one HVAC zone from another should normally be closed. (Fans of differing flow rates can cause flows across zone boundaries. This is discouraged.)

Doors that can modulate airflow within an HVAC zone should normally be in whatever position maximizes airflow to current occupants.

Reduce the amount of time personnel are allowed to be present.

Reduce the number of personnel that can be present at any one time.

As calculated by the methods given in this disclosure, assuming a generous ≈=10-hour workday, a protection factor of 4000 should be considered minimal.

### Items to be Considered at somewhat Higher Cost

Consider upgrading filter quality even if the filter shelf and nearby duct work requires modification.

On the outside of a structure, consider relocating exhaust vents and make-up (intake) air vents to avoid cross contamination between them or with other structures and their air vents.

Consider portable room air purifiers with HEPA (high-efficiency particulate air) filters.

In high-risk spaces (e.g., waiting rooms, prisons, shelters), consider UVGI (ultraviolet germicidal irradiation) that does not expose occupants.

### Under Active Debate in the Larger HVAC Community, but more Costly still

For hospitals and other infrastructure where safety is paramount, and almost any risk is too great, consider providing an external replacement to critical sections of the HVAC system. A temporary replacement can be patched into the structure on an ad-hoc basis.

### Bioprotection Methodology Based on the Lumped Element Model

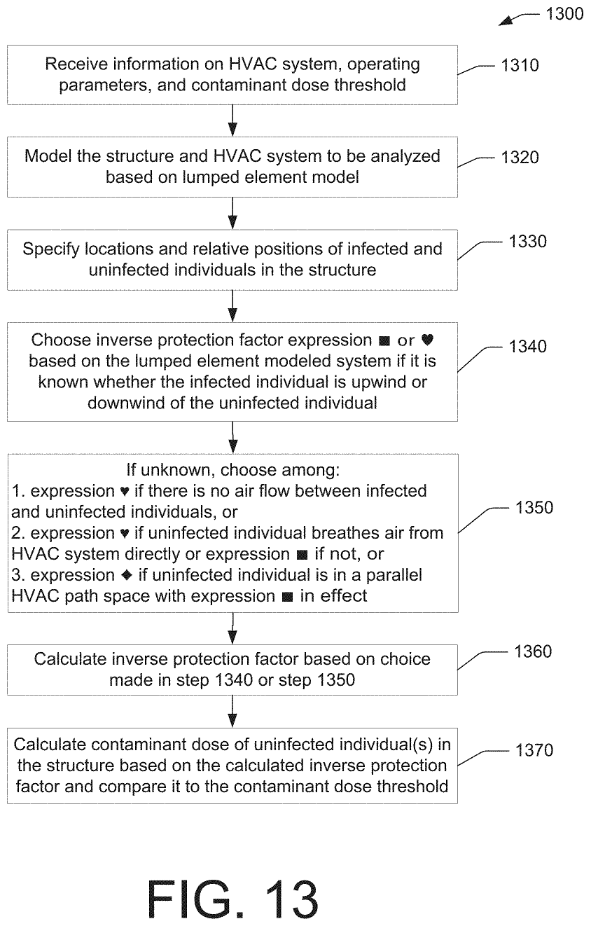

FIG. 13 shows a flow diagram illustrating one example of a process 1300 for calculating a contaminant dose based on the lumped element model. In step 1310, the process receives information on the structure (i.e., room/architectural layout) for the occupants/individuals (e.g., arrangement of spaces in series and in parallel, etc.), HVAC system layout (e.g., air intake and output, airflow in series and in parallel, internal or external release, etc.), operating parameters of the HVAC system in the structure (e.g., flow rate, air recirculation, filter transmittance, etc.), contaminant dose threshold, and the like. The contaminant dose is the amount of contaminant transferred from an infected individual to another individual and may be defined according to Eq. (F1) as the viri inhaled by the individual. The contaminant dose threshold may be preset to about a total value of 3 viri in the presence of an infected individual exhaling approximately 1200 viri per hour.

In step 1320, the process produces a modeling of the structure and HVAC system to be analyzed using the lumped element model based on the information on the structure, HVAC system, and operating parameters, and possibly input from an operator. In this specific embodiment, the inverse protection factor takes on one of two values depending on the placement of the infected and uninfected individuals. These two values are denoted as either ▪ or ♥ as presented in Eq. (F2), which is in turn based on Eqs (2) and (3) above. The inverse protection factor is calculated based on HVAC system operating parameters (the flow rate of the HVAC system R, the fraction of air recirculated η, and air filter transmittance T) and other parameters such as human parameters (e.g., average breathing rate for a human γ).

In step 1330, the process specifies locations and relative positions of infected and uninfected individuals in the structure. The inverse protection factor equation ▪ corresponds to the situation where the infected individual is upstream or upwind of the uninfected individual. The inverse protection factor equation ♥ corresponds to the situation where the uninfected individual is upstream or upwind of the infected individual. If it is known which of the infected individual and the uninfected individual is upwind, the process chooses between ♥ and ▪ as the inverse protection factor in step 1340. If this is unknown, however, the process uses the following rules in step 1350.

Rule 0: HVAC zones should be shut off from each other, so that differing pressures do not cause airflow from one zone to the next. As such, the infected individual and uninfected individual are in different HVAC zones with no airflow between and the inverse protection factor is given by the expression ♥.

Rule 1: Within a structure, if an uninfected person sits where they breathe air coming from the HVAC system directly, they are protected from an infected individual elsewhere in the structure with a protection factor associated with ♥. However, if they occupy either the same room or a room “downwind” of an infected person, the protection factor is downgraded to the quantity associated with ▪. The mass transportation situation is an example of choosing expression ▪. Choose between ♥ and ▪ based on the relative positions of the infected and uninfected individuals.

Rule 2: If an uninfected occupant is sitting where the quantity ▪ is in effect (see Rule 1), and the layout of the structure (several paths served in parallel) means that a fixed fraction of the air flows through the room containing the uninfected occupant. If this happens in the k-th room, define the fraction of airflow as σₖ. Then the expression ▪ is still used, with the quantity R is substituted with Rσₖ. This is expression ♦ of Eq. (5) above.

In step 1360, the process calculates an inverse protection factor based on the choice made in step 1340 or step 1350. Each inverse protection factor expression has a left multiplicand of γ/R and a right multiplicand that depends on T and η. For the right multiplicand, either the HVAC unit will be set up with a specified value for nor one can assume a standard value of η=0.8. If the filter is rated at a specific MERV number, the transmittance can be estimated using the second column of Table F1 in FIG. 12 or set to T=0.9 if the filter is unrated. For the left multiplicand, use Eq. (F3) above since almost all HVAC fans are rated in units of cubic feet per minute (cfm) in the United States.

In step 1370, the process calculates the expected contaminant dose of uninfected individual(s) in the structure using the lumped element model based on the calculated protection factor from step 1350 (see, e.g., Eq. (F4)) and compares the calculated contaminant dose with the preset contaminant dose threshold.

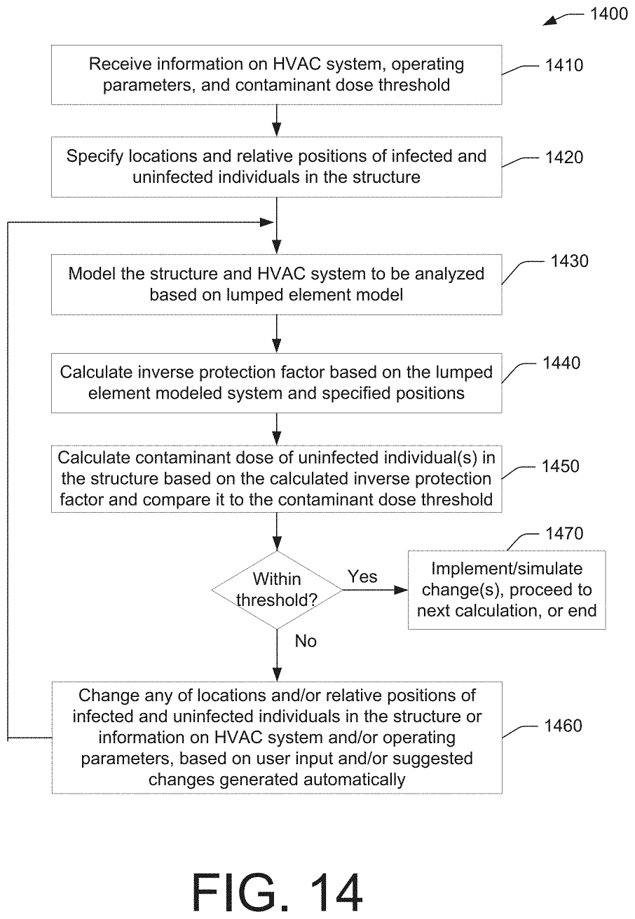

FIG. 14 shows a flow diagram illustrating an example of a bioprotection control/simulation methodology 1400 based on the lumped element model. In step 1410, the process receives information on the structure for the occupants/individuals (e.g., arrangement of spaces in series and in parallel, etc.), HVAC system (e.g., air intake and output, airflow in series and in parallel, internal or external release, etc.), operating parameters of the HVAC system in the structure (e.g., flow rate, air recirculation, filter transmittance, etc.) contaminant dose threshold, and the like. In step 1420, the process specifies locations and relative positions of infected and uninfected individuals in the structure. In step 1430, the process produces a modeling of the structure and HVAC system to be analyzed using the lumped element model based on the information on the structure, HVAC system, operating parameters, and the locations and relative positions of the individuals, and possibly with input of an operator. A part of the modeling involves determining which algebraic expression to use for the calculation of the inverse protection factor.

In step 1440, the process calculates an inverse protection factor based on the lumped element modeled system and the specified locations and relative positions of the individuals. In step 1450, the process calculates the contaminant dose of uninfected individual(s) in the structure using the lumped element model based on the calculated protection factor from step 1440 and compares the calculated contaminant dose with the preset contaminant dose threshold. The calculations may be carried out according to the algorithm using Eqs. (F1) to (F4) presented in FIG. 13 and/or using Eqs. (1) to (6) and the like described above in connection with FIGS. 4-9.

If the calculated contaminant dose is not within the preset threshold, in step 1460, the process may change any of the following: locations and/or relative positions of the infected and uninfected individuals in step 1430, or information on the HVAC system and/or operating parameters, thereby revising the modeling of the structure and HVAC system based on the lumped element model in step 1420. The changes may be based on user input and/or suggested changes generated automatically by a specially programmed computer which assesses the information on the current structure, HVAC system, operating parameters, and locations and relative positions of the individuals, and suggests changes to reduce the inverse protection factor and hence the contaminant dose based on the recommendations described in this disclosure. The process returns to step 1430 to repeat the modeling step 1430, the calculation steps 1440 and 1450, and the comparison step until the calculated contaminant dose is at or below the preset threshold.

If the calculated contaminant dose is at or below the preset threshold, in step 1470, the process may implement any changes to the HVAC system and/or operating parameters (such as the changes made in step 1460 or the recommendations discussed above) in real time, or proceed to the next calculation or simulation, or end. The next calculation may be performed for the same situation but applying different changes so that different options are available to bring the contaminant dose within the preset threshold. Implementing changes to the HVAC system can be done manually or automatically by computer control. Indeed, the steps in the bioprotection methodology of FIG. 14 may be performed manually by an operator or automatically by a specially programmed computer or via a combination of both the operator and the computer. The bioprotection methodology 1400 is used to perform assessment of the contaminant dose in real time and make changes to improve the bioprotection by reducing the contaminant dose in real time because the calculations are straightforward and can be performed quickly.

In another embodiment, the bioprotection methodology 1400 is used to design the HVAC system for the structure by performing the calculations and simulating the performance of the HVAC system. Instead of implementing physical changes to the HVAC system in step 1470, the process in design mode simulates design changes to the HVAC system until the desired inverse protection factor or contaminant dose is achieved.

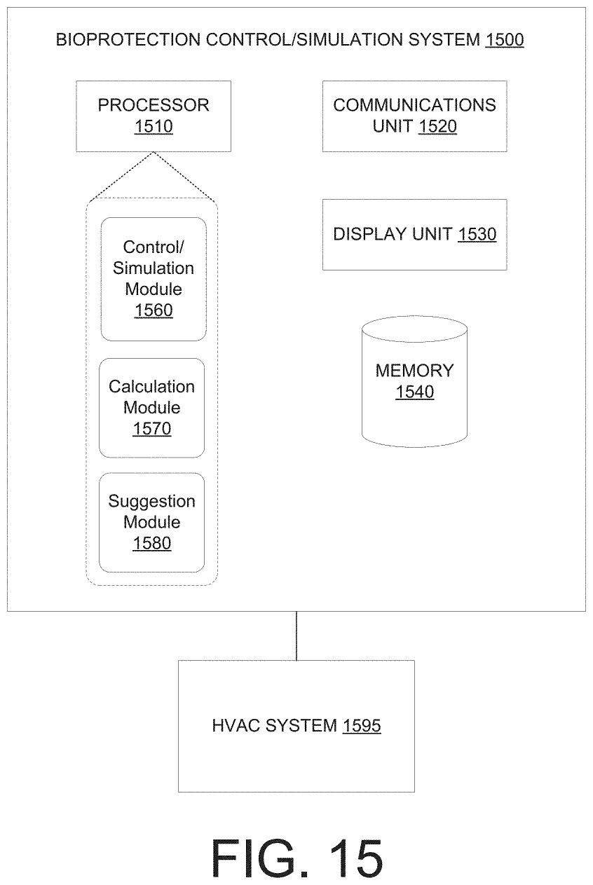

FIG. 15 is a block diagram of a bioprotection control/simulation system according to an embodiment of the invention. The bioprotection control/simulation system 1500 includes a processor 1510, a communications unit 1520, a display unit 1530, and a memory 1540. The processor 1510 may be associated with logic or modules to process information. The communications unit 1520 facilitates input/output (I/O) with one or more input or output devices including user interfaces or the like, such as keyboard, mouse, touchpad, audio and/or visual device, etc. The display unit 1530 displays information to the user. The memory 1540 stores data including input data, output data, computer instructions, and the like. Examples of input and output data include the data and parameters associated with the HVAC system as described above. Examples of computer instructions include the calculations and manipulation of data as described above and summarized in the specific embodiments of FIGS. 13 and 14. In control mode, the HVAC control system 1500 sends instructions to the HVAC system to implement changes in real time to improve bioprotection of uninfected individuals in the structure. In design mode, the HVAC design system 1500 simulates design changes to the HVAC system, for instance, until the desired inverse protection factor or contaminant dose is achieved.

Examples of modules include a bioprotection control/simulation module 1560, a calculation module 1570, and a suggestion module 1580. The bioprotection control/simulation module 1560 is executed to control or simulate changes to an HVAC system 1595. The calculation module 1570 may be configured to perform the calculations, such as those described above or shown in FIGS. 13 and 14, which can then be used by the bioprotection control/simulation module 1560 to control or simulate changes to the HVAC system. The suggestion module 1580 may be configured or programmed to suggest changes to reduce the inverse protection factor and hence the contaminant dose as described in step 1460. The suggested changes may include any changes to the structure, the HVAC system, or the placement of the individuals, and the recommendations described in this disclosure.

### Design Methodology for Bioprotection Based on the Lumped Element Model

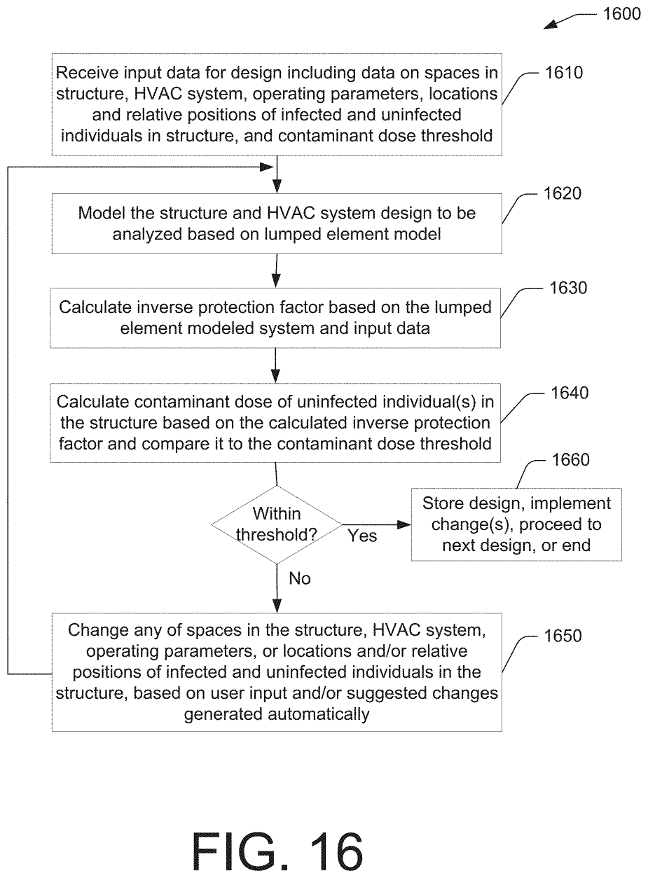

FIG. 16 shows a flow diagram illustrating an example of a bioprotection design methodology 1600 based on the lumped element model. In step 1610, the design process receives input data for the design including information on the structure for the occupants/individuals (e.g., arrangement of spaces in series and in parallel, etc.), HVAC system (e.g., air intake and output, airflow in series and in parallel, internal or external release, etc.), operating parameters of the HVAC system in the structure (e.g., flow rate, air recirculation, filter transmittance, etc.), locations and relative positions of an uninfected individual and one or more infected individuals in the structure with respect to air flowing in the structure and influenced by the HVAC system, contaminant dose threshold, and the like. In step 1620, the process produces a modeling of the structure and HVAC system to be analyzed using the lumped element model based on the information on the structure, HVAC system, operating parameters, and locations and relative positions of the individuals, and possibly with input of an operator. A part of the modeling involves determining which algebraic expression to use for the calculation of the inverse protection factor.

In step 1630, the design process calculates an inverse protection factor based on the lumped element modeled system and the specified locations and relative positions of the individuals. In step 1640, the process calculates the contaminant dose of uninfected individual(s) in the structure using the lumped element model based on the calculated protection factor from step 1630 and compares the calculated contaminant dose with the preset contaminant dose threshold. The calculations may be carried out according to the algorithm using Eqs. (F1) to (F4) presented in FIG. 13 and/or using Eqs. (1) to (6) and the like described above in connection with FIGS. 4-9.

If the calculated contaminant dose is not within the preset threshold, in step 1650, the design process may change any of the following: locations and/or relative positions of the infected and uninfected individuals or information on the HVAC system and/or operating parameters in step 1610, thereby revising the modeling of the structure and HVAC system based on the lumped element model in step 1620. The changes may be based on user input and/or suggested changes generated automatically by a specially programmed computer which assesses the information on the current structure, HVAC system, operating parameters, and locations and relative positions of the individuals, and suggests changes to reduce the inverse protection factor and hence the contaminant dose based on the recommendations described in this disclosure. The process returns to step 1620 to repeat the modeling step 1620, the calculation steps 1630 and 1640, and the comparison step until the calculated contaminant dose is at or below the preset threshold. Having the contaminant dose fall within the preset threshold is an example of having the inverse protection factor meet a preset criterion because the contaminant dose is mathematically related to the inverse protection factor (e.g., see Eq. F1 in which the contaminant dose is directly related to the inverse protection factor).

If the calculated contaminant dose is within the preset threshold, in step 1660, the design process may store the design, or if the design is for an existing structure and system, implement any of the design changes to the structure, HVAC system, operating parameters (such as the changes made in step 1650 or the recommendations discussed above), or locations and/or relative positions of the individuals, in real time, or proceed to the next design, or end. The next design may be performed for the same situation but applying different changes so that different design options are available to bring the contaminant dose within the preset threshold. Implementing changes to the HVAC system can be done manually or automatically by computer control. Indeed, the steps in the design methodology of FIG. 16 may be performed manually by an operator or automatically by a specially programmed computer or via a combination of both the operator and the computer. The design methodology 1600 is used to generate a design or make design changes to a structure and HVAC system, which can be done quickly because the calculations are straightforward and does not require numerical simulation. If the design process is performed for an existing structure and HVAC system, changes may be implemented in real time. In essence, the design changes are physically implemented to adjust the inverse protection factor to a level that meets the preset criterion for the uninfected individual in the structure having the air flowing therein and influenced by the ventilation system.

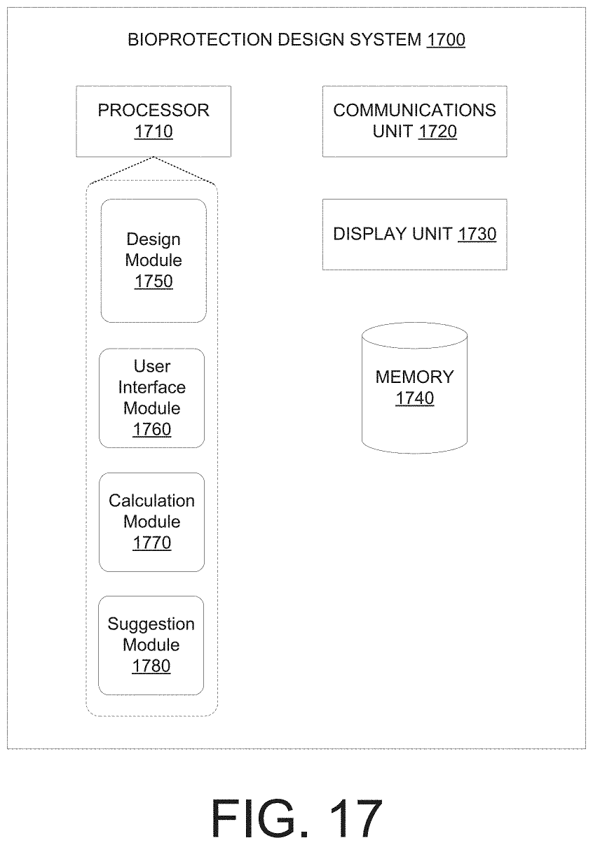

FIG. 17 is a block diagram of a bioprotection design system according to an embodiment of the invention. The bioprotection design system 1700 includes a processor 1710, a communications unit 1720, a display unit 1730, and a memory 1740. The processor 1710 may be associated with logic or modules to process information. The communications unit 1720 facilitates input/output (I/O) with one or more input or output devices including user interfaces or the like, such as keyboard, mouse, touchpad, audio and/or visual device, etc. The display unit 1730 displays information to the user. The memory 1740 stores data including input data, output data, computer instructions, and the like. Examples of input and output data include the data and parameters associated with the HVAC system as described above. Examples of computer instructions include the calculations and manipulation of data as described above and summarized in the specific embodiments of FIGS. 13 and 14. The bioprotection design system 1700 simulates design changes to the HVAC system, for instance, until the desired inverse protection factor or contaminant dose is achieved. The bioprotection system 1700 may be a computer-aided design (“CAD”) system.

Examples of modules include a bioprotection control/simulation module 1750, a user interface module 1760, a calculation module 1770, and a suggestion module 1780. The bioprotection design module 1750 is executed to simulate design of or changes to a structure, an HVAC system, operating parameters, locations and/or relative positions of infected and uninfected individuals, and the like. The user-interface module 1760 is executed to display the design and for accepting modification to the design. The design may be displayed based on input data including arrangement of spaces of the structure, operating parameters of the ventilation system, and locations and relative positions of an uninfected individual and one or more infected individuals in the structure with respect to the air flowing therein and influenced by the ventilation system.

The calculation module 1770 may be configured to perform the calculations, such as those described above or shown in FIGS. 13 and 14, which can then be used by the bioprotection design module 1750 to simulate changes to the design. The suggestion module 1780 may be configured or programmed to suggest changes to reduce the inverse protection factor and hence the contaminant dose as described in step 1650. The suggested changes may include any changes to the structure, the HVAC system, or the placement of the individuals, and the recommendations described in this disclosure.

In specific embodiments, the design module 1750 is configured to invoke the user-interface module 1760, for making the modification to the design, and to invoke the calculation module 1770 upon detecting the modification made to the design. The calculation module 1770 is configured to compare the calculated inverse protection factor to a preset criterion. An example of the preset criterion is that the contamination dose is at or below the preset contamination dose threshold. The design module 1750 is configured to invoke the user-interface module 1760, if the calculated inverse protection factor fails to meet the preset criterion, to accept a modification to the design to change at least one of the arrangement of spaces of the structure, one or more of the operating parameters of the ventilation system, or locations or relative positions of the infected and uninfected individuals in the structure, and is configured to make the modification. The design module 1750 is configured to invoke the calculation module 1770 to calculate a modified inverse protection factor for the structure based on the modification and compare the modified inverse protection factor to the preset criterion. The design module 1750 is configured to make one or more additional modifications and performing one or more additional calculations by invoking the user-interface module 1760 and the calculation module 1770 until the modified inverse protection factor meets the preset criterion.

### Computer System Example

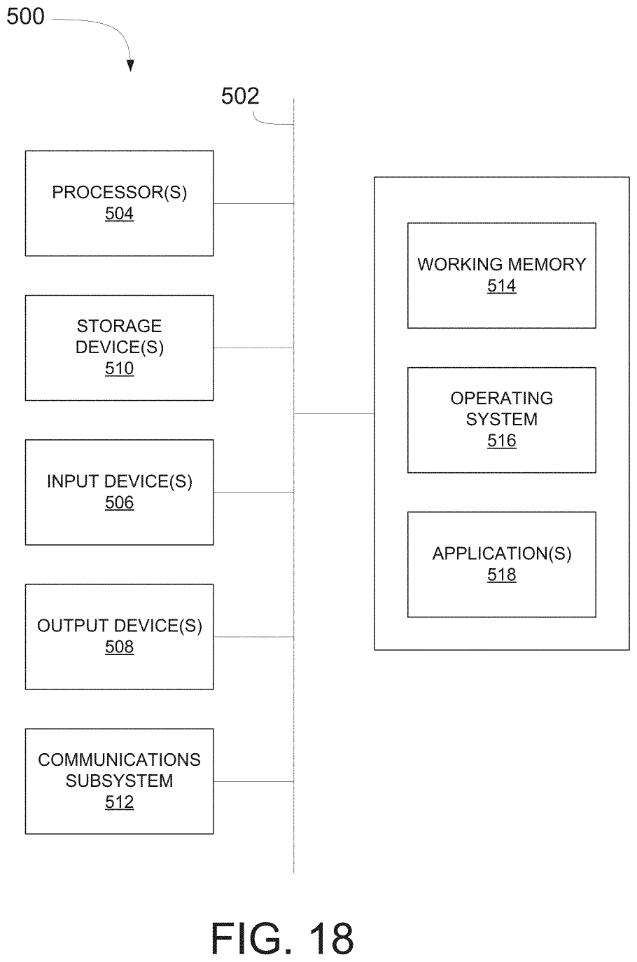

FIG. 18 depicts an example of a computer system or device configured for use with the HVAC system according to an embodiment of the present invention. The computer device 500 is shown comprising hardware elements that may be electrically coupled via a bus 502 (or may otherwise be in communication, as appropriate). The hardware elements may include a processing unit with one or more processors 504, including without limitation one or more general-purpose processors and/or one or more special-purpose processors (such as digital signal processing chips, graphics acceleration processors, and/or the like); one or more input devices 506, which may include without limitation a remote control, a mouse, a keyboard, and/or the like; and one or more output devices 508, which may include without limitation a presentation device (e.g., controller screen), a printer, and/or the like. Input to the computer system 500 may be provided by analog-to-digital converters to convert signals received from analog source or, for instance, by the input step 1410, the calculation steps 1440, 1450, the change step 1460, and the implementation step 1470 of FIG. 14, and any other measurement devices into digital form for storage and/or processing. Separate external analog-to-digital devices can be attached to the bus 502 or communication subsystem 512 to provide measurements in digital form to the computer system 500. In some cases, an output device 508 may include, for example, a display subsystem, a printer, a fax machine, or non-visual displays such as audio output devices. The display subsystem may be a cathode ray tube (CRT), a flat-panel device such as a liquid crystal display (LCD), a projection device, or the like. The display subsystem may also provide a non-visual display such as via audio output devices. In general, use of the term “output device” is intended to include a variety of conventional and proprietary devices and ways to output information from computer system 500 to a user. Output from the computer system 500 may be provided to digital-to-analog converters to send control signals from the computer to the HVAC system and any other actuators or controls used in other embodiments. Digitally controlled motors or actuators may be attached to the bus 502 or communication subsystem 512 for digital control by the computer.

The computer system 500 may further include (and/or be in communication with) one or more non-transitory storage devices 510, which may comprise, without limitation, local and/or network accessible storage, and/or may include, without limitation, a disk drive, a drive array, an optical storage device, a solid-state storage device, such as a random access memory, and/or a read-only memory, which may be programmable, flash-updateable, and/or the like. Such storage devices may be configured to implement any appropriate data stores, including without limitation, various file systems, database structures, and/or the like.

The computer device 500 can also include a communications subsystem 512, which may include without limitation a modem, a network card (wireless and/or wired), an infrared communication device, a wireless communication device and/or a chipset such as a Bluetooth device, 802.11 device, Wi-Fi device, WiMAX device, cellular communication facilities such as GSM (Global System for Mobile Communications), W-CDMA (Wideband Code Division Multiple Access), LTE (Long Term Evolution), and the like. The communications subsystem 512 may permit data to be exchanged with a network (such as the network described below, to name one example), other computer systems, controllers, and/or any other devices described herein. In many embodiments, the computer system 500 can further comprise a working memory 514, which may include a random access memory and/or a read-only memory device, as described above.

The computer device 500 also can comprise software elements, shown as being currently located within the working memory 514, including an operating system 516, device drivers, executable libraries, and/or other code, such as one or more application programs 518, which may comprise computer programs provided by various embodiments, and/or may be designed to implement methods, and/or configure systems, provided by other embodiments, as described herein. By way of example, one or more procedures described with respect to the method(s) discussed above, and/or system components might be implemented as code and/or instructions executable by a computer (and/or a processor within a computer); in an aspect, then, such code and/or instructions may be used to configure and/or adapt a general purpose computer (or other device) to perform one or more operations in accordance with the described methods.

A set of these instructions and/or code can be stored on a non-transitory computer-readable storage medium, such as the storage device(s) 510 described above. In some cases, the storage medium might be incorporated within a computer system, such as computer system 500. In other embodiments, the storage medium might be separate from a computer system (e.g., a removable medium, such as flash memory), and/or provided in an installation package, such that the storage medium may be used to program, configure, and/or adapt a general purpose computer with the instructions/code stored thereon. These instructions might take the form of executable code, which is executable by the computer device 500 and/or might take the form of source and/or installable code, which, upon compilation and/or installation on the computer system 500 (e.g., using any of a variety of generally available compilers, installation programs, compression/decompression utilities, and the like), then takes the form of executable code.

It is apparent that substantial variations may be made in accordance with specific requirements. For example, customized hardware might also be used, and/or particular elements might be implemented in hardware, software (including portable software, such as applets, and the like), or both. Further, connection to other computing devices such as network input/output devices may be employed.

As mentioned above, in one aspect, some embodiments may employ a computer system (such as the computer device 500) to perform methods in accordance with various embodiments of the disclosure. According to a set of embodiments, some or all of the procedures of such methods are performed by the computer system 500 in response to processor 504 executing one or more sequences of one or more instructions (which might be incorporated into the operating system 516 and/or other code, such as an application program 518) contained in the working memory 514. Such instructions may be read into the working memory 514 from another computer-readable medium, such as one or more of the storage device(s) 510. Merely by way of example, execution of the sequences of instructions contained in the working memory 514 may cause the processor(s) 504 to perform one or more procedures of the methods described herein.

The terms “machine-readable medium” and “computer-readable medium,” as used herein, can refer to any non-transitory medium that participates in providing data that causes a machine to operate in a specific fashion. In an embodiment implemented using the computer device 500, various computer-readable media might be involved in providing instructions/code to processor(s) 504 for execution and/or might be used to store and/or carry such instructions/code. In many implementations, a computer-readable medium is a physical and/or tangible storage medium. Such a medium may take the form of a non-volatile media or volatile media. Non-volatile media may include, for example, optical and/or magnetic disks, such as the storage device(s) 510. Volatile media may include, without limitation, dynamic memory, such as the working memory 514.

Exemplary forms of physical and/or tangible computer-readable media may include a floppy disk, a flexible disk, hard disk, magnetic tape, or any other magnetic medium, a compact disc, any other optical medium, ROM, RAM, and the like, any other memory chip or cartridge, or any other medium from which a computer may read instructions and/or code. Various forms of computer-readable media may be involved in carrying one or more sequences of one or more instructions to the processor(s) 504 for execution. By way of example, the instructions may initially be carried on a magnetic disk and/or optical disc of a remote computer. A remote computer might load the instructions into its dynamic memory and send the instructions as signals over a transmission medium to be received and/or executed by the computer system 500.

The communications subsystem 512 (and/or components thereof) generally can receive signals, and the bus 502 then can carry the signals (and/or the data, instructions, and the like, carried by the signals) to the working memory 514, from which the processor(s) 504 retrieves and executes the instructions. The instructions received by the working memory 514 may optionally be stored on a non-transitory storage device 510 either before or after execution by the processor(s) 504.

It should further be understood that the components of computer device 500 can be distributed across a network. For example, some processing may be performed in one location using a first processor while other processing may be performed by another processor remote from the first processor. Other components of computer system 500 may be similarly distributed. As such, computer device 500 may be interpreted as a distributed computing system that performs processing in multiple locations. In some instances, computer system 500 may be interpreted as a single computing device, such as a distinct laptop, desktop computer, or the like, depending on the context.

A processor may be a hardware processor such as a central processing unit (CPU), a graphic processing unit (GPU), or a general-purpose processing unit. A processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512-bit, 256-bit, 128-bit, 64-bit, 32-bit, or 16-bit data operations.

Each of the calculations or operations discussed herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described herein. All features of the described systems are applicable to the described methods mutatis mutandis, and vice versa. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like. While the exemplary embodiments have been described in some detail, by way of example and for clarity of understanding, those of skill in the art will recognize that a variety of modification, adaptations, and changes may be employed.

As will be appreciated by one of ordinary skill in the art, the present invention may be embodied as an apparatus (including, for example, a system, a machine, a device, and/or the like), as a method (including, for example, a business process, and/or the like), as a computer-readable storage medium, or as any combination of the foregoing.

Embodiments of the invention can be manifest in the form of methods and apparatuses for practicing those methods.

Unless explicitly stated otherwise, each numerical value and range should be interpreted as being approximate as if the word “about” or “approximately” preceded the value or range.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

What is claimed is:

1. A method for design of a ventilation system for a structure to be occupied by individuals, the method comprising:
accepting input data for the design including arrangement of spaces of the structure, operating parameters of the ventilation system, and locations and relative positions of an uninfected individual and one or more infected individuals in the structure with respect to air flowing in the structure and influenced by the ventilation system;
calculating an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system;
comparing the calculated inverse protection factor to a preset criterion; and
if the calculated inverse protection factor fails to meet the preset criterion, changing at least one of the arrangement of spaces of the structure, or one or more of the operating parameters of the ventilation system, or locations or relative positions of the infected and uninfected individuals in the structure, and repeating the calculating, the comparing, and the changing until the calculated inverse protection factor meets the preset criterion.

2. The method of claim 1,
wherein the inverse protection factor is an algebraic expression based on at least one of (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, (v) transmittance of an air filter used to filter airflow through the space occupied by the uninfected individual, and (vi) breathing rate of the one or more infected individuals.

3. The method of claim 1,
wherein the inverse protection factor is an algebraic expression based on (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, and (v) transmittance of an air filter used to filter airflow through the space occupied by the uninfected individual.

4. The method of claim 1, having at least one of the following three features:
wherein if the uninfected individual is upwind of the one or more infected individuals or breathes air directly from the ventilation system, then the inverse protection factor (PF⁻¹) is calculated according to
$${PF}^{- 1} = \frac{γ}{R} ( \frac{T η}{1 - T η} ) ,$$
wherein if the uninfected individual is downwind of the one or more infected individuals or does not breathe air directly from the ventilation system, then the inverse protection factor is calculated according to
$${PF}^{- 1} = \frac{γ}{R} ( \frac{1}{1 - T η} ) ,$$
and
wherein if the uninfected individual is downwind of the one or more infected individuals or does not breathe air directly from the ventilation system, and is located in a space through which only a fraction of the ventilation airflow passes, then the inverse protection factor is calculated according to
$${PF}^{- 1} = \frac{γ}{R {σ}_{k}} ( \frac{1 - T η ( 1 - {σ}_{k}}{1 - T η} ) ,$$
where
R=flow rate of the ventilation system,
η=fraction of air recirculated,
T=Transmittance of an air filter,
γ=average breathing rate for a human,
σₖ=the fraction of ventilation airflow.

5. The method of claim 1, further comprising:
calculating a contaminant dose of the uninfected individual in the structure based on the calculated inverse protection factor, according to Dose=(viri exhaled)/(Protection Factor), where the viri exhaled is an amount exhaled by the one or more infected individuals during a time period in which the uninfected individual is in the structure,
wherein the calculated inverse protection factor fails to meet the preset criterion when the calculated contaminant dose is greater than a preset contaminant dose threshold.

6. The method of claim 5,
wherein the preset contaminant dose threshold is about 3 viri in presence of an infected individual exhaling at approximately 1200 viri per hour.

7. The method of claim 1, further comprising, if the calculated inverse protection factor fails to meet the preset criterion, simulating at least one of the following changes to the design:
increasing the flow rate R of the ventilation system,
decreasing the fraction of air recirculated η,
decreasing the transmittance of the air filter,
changing the location of the uninfected individual to allow the uninflected individual to breathe air directly from the ventilation system,
changing the relative positions of the infected and uninfected individuals so that the uninfected individual is not downwind of the one or more infected individuals,
placing a partition to block airflow from the infected individual to the uninfected individual without passing through an air filter,
modifying the structure or the ventilation system to block airflow from the infected individual to the uninfected individual without passing through an air filter, or
modifying the structure or the ventilation system to produce parallel airflows through the space occupied by the uninfected individual and the space occupied by the one or more infected individuals.

8. The method of claim 7, further comprising, after the calculating, the comparing, and the changing:
physically implementing at least one of the simulated changes to adjust the inverse protection factor to a level that meets the preset criterion for the uninfected individual in the structure having the air flowing therein and influenced by the ventilation system.

9. A computer-aided design (“CAD”) system for design of a ventilation system for a structure to be occupied by individuals, the system comprising:
a user-interface module configured to display the design and to accept modification to the design, the design being displayed based on input data including arrangement of spaces of the structure, operating parameters of the ventilation system, and locations and relative positions of an uninfected individual and one or more infected individuals in the structure with respect to air flowing in the structure and influenced by the ventilation system;
a calculation module configured to calculate an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system; and
a design module configured to invoke the user-interface module, for making the modification to the design, and to invoke the calculation module upon detecting the modification made to the design;
the calculation module being configured to compare the calculated inverse protection factor to a preset criterion,
the design module being configured to invoke the user-interface module, if the calculated inverse protection factor fails to meet the preset criterion, to accept a modification to the design to change at least one of the arrangement of spaces of the structure, or one or more of the operating parameters of the ventilation system, or locations or relative positions of the infected and uninfected individuals in the structure, and being configured to make the modification,
the design module being configured to invoke the calculation module to calculate a modified inverse protection factor for the structure based on the modification and compare the modified inverse protection factor to the preset criterion, and
the design module being configured to make one or more additional modifications and performing one or more additional calculations by invoking the user-interface module and the calculation module until the modified inverse protection factor meets the preset criterion.

10. The system of claim 9,
wherein the inverse protection factor is an algebraic expression based on at least one of (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, (v) transmittance of an air filter used to filter airflow through the space occupied by the uninfected individual, and (vi) breathing rate of the one or more infected individuals.

11. The system of claim 9,
wherein the inverse protection factor is an algebraic expression based on (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, and (v) transmittance of an air filter used to filter airflow through the space occupied by the uninfected individual.

12. The system of claim 9, having at least one of the following three features:
wherein if the uninfected individual is upwind of the one or more infected individuals or breathes air directly from the ventilation system, then the inverse protection factor (PF⁻¹) is calculated according to
$${PF}^{- 1} = \frac{γ}{R} ( \frac{T η}{1 - T η} ) ,$$
wherein if the uninfected individual is downwind of the one or more infected individuals or does not breathe air directly from the ventilation system, then the inverse protection factor is calculated according to
$${PF}^{- 1} = \frac{γ}{R} ( \frac{T η}{1 - T η} ) ,$$
and
wherein if the uninfected individual is downwind of the one or more infected individuals or does not breathe air directly from the ventilation system, and is located in a space through which only a fraction of the ventilation airflow passes, then the inverse protection factor is calculated according to
$${PF}^{- 1} = \frac{γ}{R {σ}_{k}} ( \frac{1 - T η ( 1 - {σ}_{k}}{1 - T η} ) ,$$
where
R=flow rate of the ventilation system,
η=fraction of air recirculated,
T=Transmittance of an air filter,
γ=average breathing rate for a human,
σₖ=the fraction of ventilation airflow.

13. The system of claim 9,
wherein the calculation module is configured to calculate a contaminant dose of the uninfected individual in the structure based on the calculated inverse protection factor, according to Dose=(viri exhaled)/(Protection Factor), where the viri exhaled is an amount exhaled by the one or more infected individuals during a time period in which the uninfected individual is in the structure, and
wherein the calculated inverse protection factor fails to meet the preset criterion when the calculated contaminant dose is greater than a preset contaminant dose threshold.

14. The system of claim 13,
wherein the preset contaminant dose threshold is about 3 viri in presence of an infected individual exhaling at approximately 1200 viri per hour.

15. The system of claim 9, wherein the design module is configured, if the calculated inverse protection factor fails to meet the preset criterion, to make the modification to the design by invoking the user-interface module, the modification including at least one of the following changes to the design:
increasing the flow rate R of the ventilation system,
decreasing the fraction of air recirculated η,
decreasing the transmittance of the air filter,
changing the location of the uninfected individual to allow the uninflected individual to breathe air directly from the ventilation system,
changing the relative positions of the infected and uninfected individuals so that the uninfected individual is not downwind of the one or more infected individuals,
placing a partition to block airflow from the infected individual to the uninfected individual without passing through an air filter,
modifying the structure or the ventilation system to block airflow from the infected individual to the uninfected individual without passing through an air filter, or
modifying the structure or the ventilation system to produce parallel airflows through the space occupied by the uninfected individual and the space occupied by the one or more infected individuals.

16. The system of claim 9, further comprising:
a suggestion-generation module configured to provide a suggestion for a modification to the design, the suggestion based on the calculated inverse protection factor and at least one of (i) whether there is airflow between the uninfected individual and the one or more infected individuals, (ii) whether the uninfected individual is upwind or downwind of the one or more infected individuals with respect to airflow in the structure, (iii) air flow rate through a space occupied by the uninfected individual, (iv) fraction of air recirculated in the space occupied by the uninfected individual, (v) transmittance of an air filter used to filter airflow through the space occupied by the uninfected individual.

17. A computer program product for design of a ventilation system for a structure to be occupied by individuals, the computer program product embodied on a non-transitory tangible computer readable medium, comprising:
computer-executable code for accepting input data including arrangement of spaces of the structure, operating parameters of the ventilation system, and locations and relative positions of an uninfected individual and one or more infected individuals in the structure with respect to air flowing in the structure and influenced by the ventilation system;
computer-executable code for calculating an inverse protection factor for the structure using a lumped element model, the inverse protection factor being an inverse of a protection factor which is a ratio of contaminant which the one or more infected individuals exhale in the structure and contaminant which the uninfected individual inhales in the structure, the inverse protection factor being calculated based on the operating parameters of the ventilation system, the arrangement of spaces of the structure, and the locations and relative positions of the infected and uninfected individuals in the structure with respect to the air flowing therein and influenced by the ventilation system;
computer-executable code for comparing the calculated inverse protection factor to a preset criterion; and
computer-executable code for, if the calculated inverse protection factor fails to meet the preset criterion, changing at least one of the arrangement of spaces of the structure, or one or more of the operating parameters of the ventilation system, or locations or relative positions of the infected and uninfected individuals in the structure, and repeating the calculating, the comparing, and the changing until the calculated inverse protection factor meets the preset criterion.

18. The computer program product of claim 17, having at least one of the following three features:
wherein if the uninfected individual is upwind of the one or more infected individuals or breathes air directly from the ventilation system, then the inverse protection factor (PF⁻¹) is calculated according to
$${PF}^{- 1} = \frac{γ}{R} ( \frac{T η}{1 - T η} ) ,$$
wherein if the uninfected individual is downwind of the one or more infected individuals or does not breathe air directly from the ventilation system, then the inverse protection factor is calculated according to
$${PF}^{- 1} = \frac{γ}{R} ( \frac{1}{1 - T η} ) ,$$
and
wherein if the uninfected individual is downwind of the one or more infected individuals or does not breathe air directly from the ventilation system, and is located in a space through which only a fraction of the ventilation airflow passes, then the inverse protection factor is calculated according to
$${PF}^{- 1} = \frac{γ}{R {σ}_{k}} ( \frac{1 - T η ( 1 - {σ}_{k}}{1 - T η} ) ,$$
where
R=flow rate of the ventilation system,
η=fraction of air recirculated,
T=Transmittance of an air filter,
γ=average breathing rate for a human,
σₖ=the fraction of ventilation airflow.

19. The computer program product of claim 17, further comprising:
computer-executable code for calculating a contaminant dose of the uninfected individual in the structure based on the calculated inverse protection factor, according to Dose=(viri exhaled)/(Protection Factor), where the viri exhaled is an amount exhaled by the one or more infected individuals during a time period in which the uninfected individual is in the structure;
wherein the calculated inverse protection factor fails to meet the preset criterion when the calculated contaminant dose is greater than a preset contaminant dose threshold.

20. The computer program product of claim 19, wherein the preset contaminant dose threshold is about 3 viri in the presence of an infected individual exhaling at approximately 1200 viri per hour, the computer program product further comprising:
computer-executable code for, if the calculated inverse protection factor fails to meet the preset criterion, simulating at least one of the following changes to the design:
increasing the flow rate R of the ventilation system,
decreasing the fraction of air recirculated η,
decreasing the transmittance of the air filter,
changing the location of the uninfected individual to allow the uninflected individual to breathe air directly from the ventilation system,
changing the relative positions of the infected and uninfected individuals so that the uninfected individual is not downwind of the one or more infected individuals,
placing a partition to block airflow from the infected individual to the uninfected individual without passing through an air filter,
modifying the structure or the ventilation system to block airflow from the infected individual to the uninfected individual without passing through an air filter, or
modifying the structure or the ventilation system to produce parallel airflows through the space occupied by the uninfected individual and the space occupied by the one or more infected individuals.
